(19) United States
(12) Patent Application Publication
(10) Pub. No.: US 2005/0197655 A1
(43) Pub. Date: Sep. 8, 2005

(54) Method and apparatus for removing corneal tissue with infrared laser radiation and short pulse mid-infrared parametric generator for surgery

(76) Inventors: William B. Telfair, San Jose, CA (US); Hanna J. Hoffman, Palo Alto, CA (US); Henry Zenzie, Chelmsford, MA (US); Peter Moulton, Concord, MA (US); Paul R. Yoder JR., Norwalk, CT (US)

Correspondence Address: Neifeld IP Law, P.C, 4813-B Eisenhower Avenue, Alexandria, VA, 22304, US

(21) Appl. No.: 10/996,458

(22) Filed: Nov. 26, 2004

Related U.S. Application Data
(63) Continuation of application No. 09/307,988, filed on May 10, 1999.
Application No. 09/307,988 is a continuation-in-part of application No. 08/549,385, filed on Oct. 27, 1995, now Pat. No. 5,782,822.

(51) Int. Cl. A61B 18/20

(52) U.S. Cl. 606/5; 606/10

(57) ABSTRACT

A surgical technique for removing corneal tissue with scanned infrared radiation is disclosed which utilizes short mid-infrared laser pulses to provide a tissue removal mechanism based on photospallation. Photospallation is a photomechanical ablation mechanism which results from the absorption of incident radiation by the corneal tissue. Since photospallation is a mechanical ablation process, very little heat is generated in the unablated adjacent tissue. The disclosed surgical system includes a scanning beam delivery system which allows uniform irradiation of the treatment region and utilizes low energy outputs to achieve controlled tissue removal. A real-time servo-controlled dynamic eye tracker, based on a multiple-detector arrangement, is also disclosed which senses the motion of the eye and provides signals that are proportional to the errors in the lateral alignment of the eye relative to the axis of the laser beam. Temporal and frequency discrimination are preferably utilized to distinguish the tracking illumination from the ambient illumination and the surgical laser beam. A laser parametric generator for surgical applications is disclosed which utilizes short-pulse, mid-infrared radiation. The mid-infrared radiation may be produced by a pump laser source, such as a neodymium-doped laser, which is parametrically down converted in a suitable nonlinear crystal to the desired mid-infrared range. The short pulses reduce unwanted thermal effects and changes in adjacent tissue to potentially submicron-levels. The parametrically converted radiation source preferably produces pulse durations shorter than 25 ns at or near 3.0 microns but preferably close to the water absorption maximum associated with the tissue. The down-conversion to the desired mid-infrared wavelength is preferably produced by a nonlinear crystal such as KTP or its isomorphs. In one embodiment, a non-critically phased-matched crystal is utilized to shift the wavelength from a near-infrared laser source emitting at or around 880 to 900 nm to the desired 2.9-3.0 microns wavelength range. A fiber, fiber bundle or another waveguide means utilized to separate the pump laser from the optical parametric oscillation (OPO) cavity is also included as part of the invention.

### CROSS—REFERENCE TO RELATED APPLICATIONS

This application is a continuation of application Ser. No. 09/307,988, filed May 10, 1999, which is a continuation-in-part of patent application Ser. No. 08/549,385, filed Oct. 27, 1995. Application Ser. No. 09/307,988 has attorney docket number VISX0011U/US, and Ser. No. 08/549,385 has attorney docket number VISX0017U/US. The disclosures of both of the foregoing applications are incorporated herein by reference.

### FIELD OF THE INVENTION

The present invention relates to laser surgical techniques for modifying the corneal surface of the eye, and more particularly, to laser surgical techniques, collectively known as photorefractive keratectomy or PRK, which direct reshaping of the cornea by means of selective volumetric removal of corneal tissue.

### BACKGROUND OF THE INVENTION

In recent years, numerous corneal sculpting techniques and related apparatus have been disclosed for correcting visual deficiencies such as near-sightedness, far-sightedness, and astigmatism. In addition, corneal sculpting techniques have also been utilized for therapeutic interventions in a number of pathologic conditions involving the cornea. For example, U.S. Pat. Nos. 4,665,913, 4,732,148, and 4,669,466 to L'Esperance, and U.S. Pat. No. 5,108,388 to Trokel, describe methods for achieving optical correction through reshaping of the anterior corneal surface. In addition, a number of prototype instruments for affecting refractive surgery have recently become commercially available, such as the Model 2020 from Visx of Santa Clara, Calif. and the Model Exci-Med 200 from Summit of Watertown, Mass.

These commercial devices, as well as most corneal sculpting methods and devices which have been disclosed and manufactured to date, utilize ultraviolet (UV) radiation with a wavelength which is preferably less than 200 nanometers. For example, many of these devices utilize an Argon Fluoride excimer laser operating at 193 nm. Generally, radiation at such short ultraviolet wavelengths is characterized by high photon energy, namely, greater than 6 eV, which, upon impact with tissue, causes molecular decomposition, i.e., the direct breaking of intramolecular bonds. The photochemical nature of this mechanism has the advantage of producing minimal collateral thermal damage in cells adjacent to the surgical site, since the broken molecules generally leave behind only small volatile fragments which evaporate without heating the underlying substrate. Furthermore, the depth of decomposition for each laser pulse is typically very small, i.e., less than 1 micron, thus achieving accurate tissue removal with minimal risk of damage to the underlying structures from UV radiation.

In view of this small depth of penetration, coupled with the need to remove sufficient depth of tissue while minimizing the overall time for the surgical procedure, the majority of corneal sculpting techniques utilizing the excimer laser employ “wide area ablation”. Generally, wide area ablation utilizes a laser beam with a relatively large spot size to successively remove thin layers of corneal tissue. The spot size is generally of a size sufficient to cover the entire optical zone of the cornea, namely, a region of 5 to 7 millimeters in diameter. Consequently, to assure required flux densities on the cornea, relatively high energy output UV lasers are typically required. It has been found that to assure a flux density of at least 150 mJ per square centimeter, for a reasonable ablation rate of at least 0.2 microns/pulse, a 200 mJ UV laser is required. Such lasers, however, tend to be prohibitively large and expensive systems.

Furthermore, efficacious wide area ablation requires that the projected beam be spatially homogenous and uniform to achieve the desired smooth corneal profiles. Accordingly, additional beam shaping devices, such as rotating prisms, mirrors, or spatial integrators, must be employed within the excimer beam delivery systems. For a more detailed discussion of beam shaping and delivery systems, see, for example, U.S. Pat. No. 4,911,711 to Telfair, incorporated by reference herein. Of course, such a multiplicity of optical elements contributes to overall transmission loss, while adding substantial optical complexity, cost, and maintenance requirements to the system.

Alternative techniques based on utilization of a scanning UV laser beam have been proposed to achieve controlled and localized ablation of selected corneal regions of the cornea. In the scanning approach, a relatively small laser spot is scanned rapidly across the cornea in a predefined pattern and accumulatively shapes the surface into the desired geometry. For a more detailed discussion of laser scanning techniques employing excimer lasers, see U.S. Pat. No. 4,665,913 to L'Esperance or Lin, J. T., “Mini-Excimer Laser Corneal Reshaping Using a Scanning Device,” SPIE Proceedings, Vol. 2131, Medical Lasers & Systems III (1994). A scanning approach may offer a number of advantages, including lower power and energy requirements, added flexibility for refractive corrections and smooth ablation profiles, without the need for spatially uniform output beam profiles. For example, a laser scanning technique allows a tapered optical treatment zone to be achieved, which may have advantages for the correction of high myopia, for performing therapeutic tissue removal and for treating areas up to 9 millimeters in diameter which may be required for the correction of hyperopia.

While laser surgical techniques based on the excimer laser have proved beneficial for many applications, such techniques suffer from a number of limitations, which, if overcome, could significantly advance the utility of optical laser surgery. For example, techniques based on excimer lasers utilize toxic gases as the laser medium, suffer from persistent reliability problems, require lossy optics in the delivery systems, and suffer from the possibility that the UV radiation is potentially mutagenic through secondary fluorescence, which may cause undesirable long term side effects to the unexposed tissues of the eye.

Accordingly, alternatives to the excimer laser have been suggested in recent years which involve frequency-shifted radiation from a solid state laser. Current limitations of nonlinear elements used as frequency-shifting devices, however, place a lower limit of approximately 205 nm on the available wavelengths of such lasers, which may be too close to the mutagenic range, which exhibits a peak at 250 nm. In addition, multiply-shifted laser devices also face certain difficulties in providing the requisite energy outputs and are fairly complex and cumbersome, leading again to potential laser reliability problems, as well as added cost and maintenance.

More recently, a more attractive alternative has been suggested by T. Seiler and J. Wollensak, “Fundamental Mode Photoablation of the Cornea for Myopic Correction”, Lasers and Light in Ophthalmology, 5, 4, 199-203 (1993), involving mid-infrared wavelengths and, in particular, radiation around 3 microns corresponding to the absorption peak of water, the main constituent of the cornea. One solid state laser in particular, the Erbium:YAG laser (Er:YAG), emits radiation at a wavelength of 2.94 microns, corresponding to an absorption coefficient of over 13000 per centimeter in water. This high absorption results in a small region of impact with potentially less than two micron penetration depths.

Contrary to the photoablation mechanism associated with the excimer laser, i.e., photochemical decomposition, which is due to energy absorption in molecular bonds, ablation with the Er:YAG laser is attributed to photovaporization, or photothermal evaporation, of water molecules. This thermal heat induces a phase change, and thus a sudden expansion of the tissue material, thereby ablating the corneal surface tissue.

In addition, erbium lasers are more attractive for clinical applications than excimer lasers, since they are compact, efficient and can deliver higher beam quality radiation, which allows for less lossy beam delivery systems and superior optical coupling properties. Further, the photovaporization process is inherently more efficient than photodecomposition, allowing for removal of up to 3 microns of tissue at a time and thereby resulting in a faster surgical operation. Mid-infrared radiation is also compatible with fiber delivery, a potentially attractive method of decoupling the source laser from the delivery system which makes it more suitable for the operating room. Finally, radiation from an Er:YAG laser is not mutagenic, relieving the potential for deleterious long-term side effects.

The Er:YAG laser-based corneal sculpting system described by Seiler and Wollensak is based on wide area ablation. This system aims to exploit the gaussian beam profile of the laser beam to achieve a refractive correction with each pulse, using a minimal number of pulses. An alternative system which also relies on wide area ablation is described in PCT Application No. 93/14817 to Cozean et al., which relies on a sculpting filter to control the intensity of the radiation delivered to the cornea and hence the amount of tissue removal.

While providing a number of advances over prior techniques, the Er:YAG laser techniques described by Seiler and Wollensak and Cozean et al. both suffer from a number of potential drawbacks, common to wide area ablation techniques, including the need for a smooth and uniform beam profile, a large pulse energy, and/or a complex filter control system. These systems assumed that the ablation process is a linear process, i.e., that a portion of the beam with a larger energy density will remove a larger depth of tissue. This has been shown, however, to be an incorrect assumption for the excimer ablation process, and may also be an incorrect assumption for the Er:YAG ablation process.

In addition to the limitations previously discussed, all such prior techniques for delivering and controlling a mid-infrared laser beam are subject to one shortcoming in particular, namely, the potential for thermal damage to unablated regions of the eye, due to excessive energy density required by these systems and the large shock waves generated by the high energy pulses required to ablate wide areas. In addition, due to the need for high pulse energy and high beam quality, such prior systems typically exhibit optical configurations that are generally not conducive to ease of manufacturing and are difficult to maintain and service.

As is apparent from the above discussion, a need exists for an improved method and apparatus for surgically treating corneal tissue based on the controlled removal of tissue. A further need exists for an improved method and apparatus for reducing myopic, hyperopic and/or astigmatic conditions of the eye using a low cost solid state laser. Yet another need exists for a method and computer-controlled apparatus for scanning mid-infrared laser radiation across the outer surface of the eye and the underlying Bowman's layer and stroma for the purpose of reducing refractive errors of the eye and for the purpose of treating tissue at or near the surface of the cornea. A further need exists for a method and apparatus for surgically treating corneal tissue, having an improved eye tracking mechanism.

In recent years, photorefractive keratectomy (PRK) techniques for reshaping the cornea of the eye have become widely utilized as an effective means for correcting visual deficiencies. These methods are generally based on volumetric removal of tissue using ultraviolet (UV) radiation, typically from a 193 nm ArF excimer laser. At this short wavelength, the high photon energy causes direct breaking of intramolecular bonds, in a process known as photochemical decomposition. Tissue ablation based on this photochemical mechanism has the advantage of producing minimal collateral thermal damage in cells adjacent to the surgical site. Also, the depth of decomposition is very small, typically less than 1 micron, resulting in accurate tissue removal with minimal risk of damage to underlying structures from UV radiation.

While excimer-based methods have been established as a safe and effective method of corneal ablation, they suffer from a number of deficiencies, including high initial cost and ongoing maintenance costs, large and complex optical beam delivery systems, safety hazards due to the fluorine and ozone gas formation and persistent reliability problems. Furthermore, the potential phototoxicity of high-power UV radiation is still an undetermined risk in excimer-laser-based PRK. In particular, there is concern that the UV radiation poses certain mutagenic and cataractogenic risks due to secondary fluorescence effects.

A recently suggested alternative to the excimer laser for performing corneal refractive surgery involves ablation at mid-infrared wavelengths using, in particular, radiation around 382 m corresponding to the absorption peak of water, the main constituent of the cornea. The premise underlying interest in such an alternative system is that infrared radiation can be produced with solid-state technology, which would provide easier handling, is cheaper, more compact and has better reliability features while eliminating the potential of any safety concerns due to toxic gases or mutagenic side effects associated with UV wavelengths. One solid state laser in particular, the erbium:YAG (Er:YAG) laser, emits radiation at a wavelength of 2.94 microns, corresponding to an absorption coefficient of over 13000 per centimeter in water. This high absorption results in a relatively small region of impact with potentially less than 2 microns penetration depth. Contrary to the photoablation mechanism associated with the excimer laser, i.e., photochemical decomposition, ablation at the erbium wavelength is attributed to photovaporization, or photothermal evaporation, of water molecules. This process is inherently more efficient than photodecomposition, allowing for removal of up to 3 microns of tissue at a time, resulting in faster surgical operation. Such a system has been suggested for example by T. Seiler and J. Wollensak, “Fundamental Mode Photoablation of the Cornea for Myopic Correction”, Lasers and Light in Ophthalmology, 5, 4, 199-203 (1993). Another system has been described by Cozean et al. in PCT Application No. 93/14817, which relies on a sculpting filter to control the amount of tissue removal using a pulsed 3 micron Er:YAG laser. However, while ophthalmic surgical techniques based on free running or long-pulse erbium lasers have shown some promise, they also suffer from a number of drawbacks principally relating to the fact that the IR radiation causes collateral thermal damage to tissue adjacent to the ablated region, where the size of the damage zone may exceed several microns, resulting in potentially undesirable long term effects.

Recently, it has been recognized that lasers having a pulse duration shorter than a few tens of nanoseconds will demonstrate less dominant thermal effects. In particular, a direct tissue interaction effect known as photospallation has been observed at infrared wavelengths whereby, with shorter pulses, radiation interacts exclusively with the irradiated tissue producing negligible effect upon the adjacent, unirradiated tissue. Photospallation is a photomechanical ablation mechanism which results from the rapid absorption of incident radiation and subsequent expansion by the corneal tissue. This expansion is followed by a bi-polar shock wave that causes removal of tissue. For a detailed description of a method and apparatus for performing corneal surgery that directly exploits the photospallation mechanism to remove tissue, see U.S. patent application Ser. No. 08/549,385, the parent application to the present invention, which is incorporated by reference herein. The method and apparatus disclosed therein utilize a short-pulse (preferably less than 50 ns) solid state laser emitting mid-infrared radiation, preferably at or around 2.94 microns, scanned over a region of the cornea to allow uniform irradiation of the treatment region using a relatively low-energy laser. As pointed out in the parent application, a desired laser source for this application would have output energy of up to 30 mJ and repetition rates of up to 100 Hz, depending on the details of the delivery system.

An erbium-doped laser operating at 2.94 microns is one option for such a laser source. A compact, reliable Q-switched erbium laser is described in our co-pending patent application Ser. No. 08/549,385. While highly attractive because of its simplicity, even with the aid of future diode pumping, it may be difficult to extend the erbium laser operation to high repetition frequencies (in excess of 30 Hz) due to strong thermal birefringence effects. Limitations of the fundamental level dynamics and long upper-laser-level lifetimes may also conspire with peak-power damage to optical component coatings to impose a practical lower limit on the pulse duration of 20 ns or so in an erbium-based laser operating in a Q-switched mode.

Recognizing that it is possible that a shorter pulse (less than 20 ns) may increase the percentage of true photospallative ablation process, and thus further reducing residual contributions to tissue ablation from undesirable thermal effects, it is desirable to construct the shortest pulse solid state mid-infrared laser source that can safely and efficaciously meet the requirements of PRK. Ideally, such a source would also be scalable to high repetition frequencies (approaching 100 Hz) without substantially increasing the expense and complexity of the device or compromising its reliability.

An Optical Parametric Oscillator (OPO) that can downshift the frequency of radiation from a standard neodymium-doped laser, such as Nd:YAG, operating at or about 1.06 microns has been suggested as an alternative approach in our co-pending U.S. patent application Ser. No. 08/549,385, to obtaining the desired parameters at mid-IR wavelengths. However, no such device has been available to date that can meet all the requirements of the ophthalmic surgical procedures contemplated. For example, efficient OPOs which are pumped by a 1 micron laser with output in the IR range have been demonstrated in recent years using a number of different nonlinear crystals such as Lithium Niobate (LiNbO₃) and Potassium Titanyl Phosphate (KTiOPO₄ or “KTP”). Examples of parametric oscillation near the 3 microns wavelength of interest include the generation of high-power radiation (8 W) at 3.5 microns using LiNbO₃ pumped by a 100 Hz, single-mode pump beam (see A. Englander and R. Lavi, OSA Proceedings on Advanced Solid-State Lasers, Memphis, Term., 1995, p. 163) and demonstration of a 0.2 W output at 3.2 microns using KTP in a non-critical phase match configuration (see, for example, K. Kato in IEEE J. Quantum Electronics. 27, 1137 (1991)). Realization of an optical parametric device with output at the desired 2.9 to 3.0 microns wavelength range was considered difficult because the two readily available candidate crystals of LiNbO₃ and KTP exhibit absorption in that wavelength range. Use of LiNbO₃ in particular is not considered feasible because of absorption at or near 3.0 microns due to the OH-band present in the crystal using current growth methods. Other drawbacks of the OPO design include a perceived requirement for powerful and high-beam-quality pump sources that can overcome the high threshold for the onset of a parametric process. Since the effectiveness of increasing the pump power density by focusing the pump beam is limited by the walk-off angle of the nonlinear crystal, the threshold condition cannot be overcome simply by using small pump beam diameters in most crystals. A way to circumvent this problem is to use a crystal that can be non-critically phase-matched (such as KTP), resulting in higher acceptance angles, but this configuration is not possible for a 1 microns pump beam wavelength and with the output wavelength desired for a successful PRK procedure. Non-critical phase-matching with output in the 2.9-3.0 microns range is, however, feasible in KTP (x-cut) pumped at 0.88 to 0.9 microns. Lasers emitting at this wavelength range are, however, more complex and expensive than standard neodymium doped laser at or near 1 micron.

For a medical laser instrument, it is generally not desirable to impose overly stringent requirements on the pump laser, as that would result in more complex and costly systems. Ideally, a multimode gaussian or a top-hat beam profile that is commercially available would be desired. However, prior to the present invention, it was not clear that such a pump beam, which can possess substantial divergence, would produce the requisite output energies without damaging the OPO crystal and/or the coupling optics. Also, in the case of a gaussian spatial profile beam, uneven distribution of the peak power density across the crystal can result in only part of the beam contributing significantly to the parametric generation thereby compromising the efficiency of conversion. Furthermore, absorption in KTP, which is known to be substantial at 3.0 microns, was another issue of concern especially for operation at elevated average power levels and/or high repetition rates. These as well as other reasons prevented the realization to date of an OPO source of pulsed 2.9-3.0 microns radiation of practical output energies and repetition rates.

The present invention discloses a specific apparatus for producing short-pulse radiation at or near 2.94 microns which overcomes the aforementioned difficulties. The apparatus is uniquely suited to performing PRK and other microsurgery procedures at minimal complexity and low cost, thus greatly increasing the availability of such procedures to a large number of people. Furthermore, with certain adjustments to the apparatus, it may be used for certain other ophthalmic procedures where a concentrated pulsed beam at a selected mid-IR wavelength has demonstrated benefits. These procedures include laser sclerostomy, trabeculectomy and surgery of the vitreous and/or the retina. In these procedures means for affecting precise, highly localized tissue ablation are desired. For example, in the case of laser-assisted vitroretinal surgery, the application of mid-IR radiation at 2.94 microns offers the potential of tractionless maneuvers, shallow penetration depths and extreme precision both in transecting vitreous membranes and in ablating requisite epiretinal tissue. See, for example, J. F. Berger, et al. in SPIE, vol. 2673, 1994, p. 146. Furthermore, by utilizing short pulses as disclosed in the present invention, the procedure may be efficaciously conducted at lower fluence levels thus easing requirements on probe geometry. In glaucoma filtration procedures such as ab externo sclerostomy, where a fistula is created from the anterior chamber of the eye into the subconjuctival space, the application of a nanosecond, low energy pulses from an excimer laser at 308 mm proved highly advantageous in treating a number of severely affected patients. See, for example, J. Kampmeier et al. in Ophthalmolge, 90, p. 35-39, 1993. Similar effectiveness of the procedure is expected for mid-IR wavelength due to the high absorption properties of the sclera. The main issue which prevented wider use to date of mid-IR laser radiation in micro-ocular surgery was the lack of a suitable fiber for delivering the energy to the target tissue. However, recent developments in this area culminated in a number of potential fiber technologies including zirconium fluoride, sapphire silver halide and hollow waveguide technologies. With further improvements in damage thresholds, it appears that sufficiently flexible, low loss fibers and appropriate probes may become available in the very near-term that can handle delivery of even short pulse, 3-micron radiation, for lower energy (<20 mJ) applications. The emergence of such fiber delivery systems may also make short pulse, mid-IR radiation highly attractive in general endoscopic microsurgery. In particular, medical procedures such as brain, orthoscopic and spinal cord surgery may benefit from the highly localized effects generated by the photo-mechanical ablation associated with the present system because the delicate nature of the tissues involved places a premium on limiting collateral thermal injury in surrounding tissue. Of course, optimal parameters of the laser may very with the application, tissue type and desired effect. But in this respect, the OPO laser has an advantage in that it offers great flexibility in terms of available outputs including variations in wavelength and pulse duration.

### SUMMARY OF THE INVENTION

Generally, according to aspects of the invention, a surgical method and apparatus for removing corneal tissue with mid-infrared radiation are provided. The surgical method and apparatus utilize short laser pulses scanned over a region of the cornea to yield a tissue removal mechanism based on photospallation. Photospallation is a photomechanical ablation mechanism which results from the absorption of incident radiation by the corneal tissue. When the corneal tissue absorbs the infrared radiation, a bipolar oscillating shock wave is created, which alternately compresses and stretches the corneal tissue. Tissue fragments are torn apart and ejected by the shock wave during the stretching phase.

In accordance with one feature of the present invention, the laser delivery system includes a laser source, such as a Q-switched Er:YAG laser, which emits pulsed radiation in the mid-infrared spectral region with an energy density capable of causing ablation of corneal tissue. In a preferred embodiment, the laser emits radiation of approximately 3 microns, corresponding to the maximal absorption coefficient of water, the main constituent of corneal tissue. The laser source preferably emits radiation at discrete pulses of less than 50 nanoseconds at a repetition rate of approximately 5 to 100 Hertz. The short laser pulses reduce the undesirable thermal damage of surrounding tissue to insignificant levels. The energy in each pulse is preferably on the order of 5 to 30 mJ.

The laser beam is preferably scanned over a specific central region of the surface of the cornea in a predefined pattern by a scanning beam delivery system so as to selectively remove tissue at various points within the scanned region and thereby reshape the corneal tissue in a predictable and controlled fashion. The scanning beam delivery system preferably consists of a controllable tilt mirror assembly to direct and aim the beam over the surface of the cornea. A variety of predefined scan patterns may be utilized to achieve controlled photospallation of the cornea, including the epithelium, Bowman's layer, and the stroma in accordance with the desired changes in the shape of the cornea.

In accordance with a further aspect of the present invention, the laser spot size and spacing associated with a given scan pattern may be varied prior to each procedure according to certain nomograms correlating the required degree of pulse overlap with the depth of ablation, consistent with maximizing the speed of the operation and the requisite smoothness of the ablated corneal surface. A given scan pattern preferably uniformly irradiates a treatment region with minimal discernible lines of overexposed or underexposed tissue lying between scans. One or more discontinuous scan patterns may be utilized to distribute the pulse over the entire treatment region in each time interval, thereby distributing residual heat over the entire region and minimizing temperature rise in any localized area.

Further, in accordance with a preferred embodiment of the invention, an eye tracking system is further provided in conjunction with the scanning beam delivery system, to compensate for eye motion during the surgical procedure. The eye tracking system senses the motion of the eye and provides signals that are proportional to the errors in lateral alignment of the eye relative to the axis of the laser beam. Lateral motion of the eye is detected by illuminating the eye with tracking illumination and forming an image of a significant feature of the eye, such as the limbus, on an array of detectors. According to a feature of the present invention, the array of detectors includes at least four detectors centered vertically and horizontally around the center of the detector array.

In operation, when the significant feature of the eye is centered with respect to the axis of the laser beam, the image of the significant feature will be centered on the detector array. A null signal is generated by the detector array which serves to maintain the axis of the laser beam in its current position. When the eye is not centered with respect to the axis of the laser beam, however, the image formed on the detector array will also not be centered. The detector array will generate an error signal which causes the laser beam to be deflected to ensure that it is properly applied to the corneal tissue.

The tracking illumination is preferably chosen in the near infrared range so that it may be discriminated from ambient illumination and the laser beam. In addition, the tracking illumination is preferably modulated at a predefined temporal frequency to further discriminate the tracking illumination from the ambient illumination and the laser beam. Red or near infrared filters may be positioned in front of the detectors in the array to further enhance the contrast of the significant feature of the eye to be detected, such as the limbus.

According to further features of the invention, a corneal topography device may be included in the surgical apparatus for evaluating the shape of the corneal tissue to assist in pre-op or post-operative measurements. Alternatively, a spatially resolved refractometer may be included for evaluating the refraction of the corneal tissue. In various embodiments of the invention, the above-described alignment methods may be utilized to incorporate active feedback control from the topographic or refraction mapping instrument so as to provide further control over the course of the surgical procedure.

A more complete understanding of the present invention, as well as further features and advantages of the invention, will be obtained by reference to the detailed description and drawings.

It is therefore an object of this invention to provide a new and improved surgical apparatus, that is particularly adapted for performing corneal refractive surgery. It is another object to facilitate a new and improved method of photorefractive laser surgery based on utilizing short-pulse, mid-infrared radiation produced by parametric downconversion of radiation from a neodymium-doped laser, such as Nd:YAG.

The short pulses are viewed as critical to reducing unwanted changes in adjacent tissue and especially thermal effects which can result in undesirable irregular edges of the interaction site produced by the infrared radiation. With sufficiently short pulses, the thermal damage can be reduced to potentially sub-micron levels, resulting in the same clinical indications as ablative photodecomposition produced by deep-UV lasers, commonly used in refractive surgical procedures. Consequently, it is a key aspect of the present invention to provide a laser source with pulse durations shorter than 25 ns at or near 3.0 microns but preferably close to the 2.94 microns water absorption maximum.

It is a further object of this invention to provide a new and improved laser surgical apparatus utilizing an OPO based on a nonlinear crystal such as KTP or its isomorphs for shifting the wavelength of a neodymium-doped laser to the desired mid-infrared wavelength range near 3.0 microns. In an alternative embodiment, a related objective would be to provide a non-critically phased-matched crystal to shift the wavelength from a near-infrared laser source emitting at or around 880-900 nm to the desired 3.0 microns wavelength range.

In yet another object, the OPO cavity parameters are such as to accommodate a readily available pump beam of moderate power while still producing a stable output with pulse energies scalable to the tens of millijoules level. In a preferred embodiment of the OPO laser, pump beams that are single or multi-mode with either gaussian or top-hat spatial profiles and with divergence ranging to many times the diffraction limit would all be accommodated, while maintaining a simple optical configuration with a minimum number of elements.

It is a further object to provide, within the OPO configuration, means for elevating damage thresholds, such that short pulse pump beams with energy outputs over 200 mJ at wavelengths at or near 1-micron can all be accommodated without damage at repetition rates exceeding 10 Hz and preferably approaching 50 Hz. A related object is to provide optimal OPO configurations such that the lowest pump thresholds result for a desired output in the mid-IR range.

It is still another object to provide a new apparatus and method for performing refractive surgery using a fiber or a fiber bundle or some other waveguide means to separate the pump laser from the OPO cavity. The OPO portion could then be mounted to the surgical microscope providing the surgeon with maximal flexibility for delivering the light to the patient's eye.

A more complete understanding of the present invention, as well as further features and advantages of the invention, will be obtained by reference to the detailed description and drawings.

### BRIEF DESCRIPTION OF THE DRAWINGS

FIG. 1 is a block diagram illustrating the functional relationship of optical, mechanical, and electrical components of apparatus incorporating features of the present invention;

FIG. 2 is an expanded schematic diagram of the optical components of FIG. 1;

FIGS. 3(a) and 3(b) illustrate scanning patterns for the laser beam passing over the cornea;

FIGS. 4(a) and 4(b) illustrate intensity profiles as a function of the diameter of the focused laser beam, measured at the cornea;

FIGS. 5(a) and 5(b) illustrate mechanisms for transferring the laser beam from the laser system to the surgical apparatus;

FIGS. 6(a) and 6(b) illustrate images of the eye in an aligned and unaligned position, respectively, with respect to the detector array of an integral eye tracker; and

FIG. 7 is a schematic diagram of one embodiment of the electronic circuitry and servo control functions associated with the eye tracker indicated in FIGS. 1 and 2.

FIG. 8 is a schematic diagram illustrating a preferred embodiment of the OPO laser device according to the present invention.

FIG. 9 is a schematic diagram illustrating an alternative embodiment of the OPO laser source, using an L-shaped configuration.

FIG. 10 is a schematic diagram illustrating another alternative embodiment of the OPO using a single-pass pump beam.

FIG. 11 is a schematic diagram illustrating yet another alternative embodiment using single-pass pump beams in a ring configuration.

FIG. 12 is a schematic diagram illustrating of a preferred embodiment of the OPO laser source where the pump beam is fiber coupled to the OPO.

### DETAILED DESCRIPTION

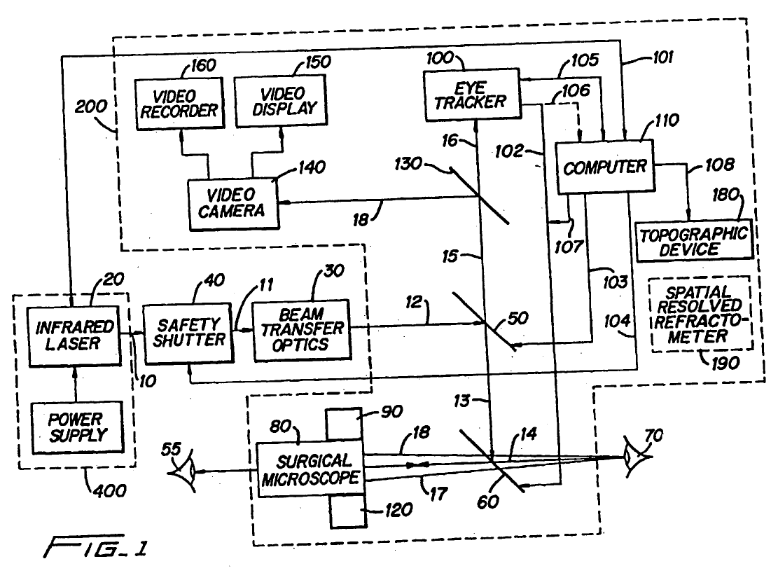

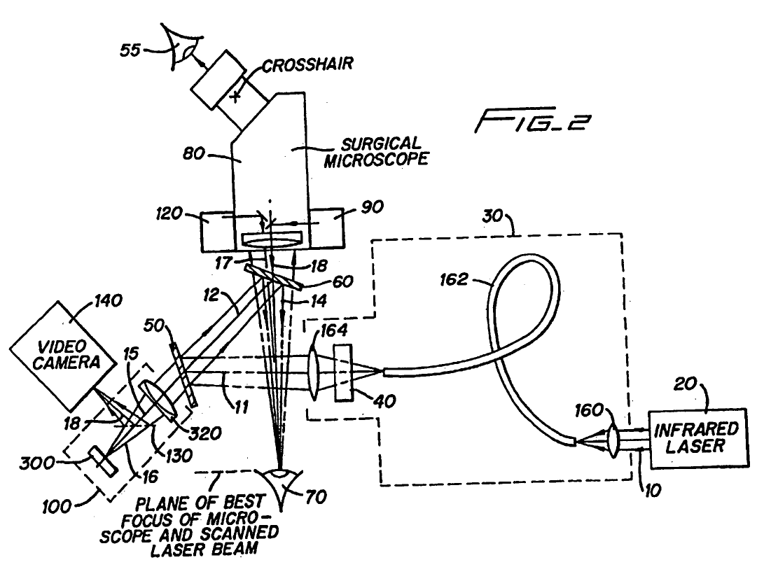

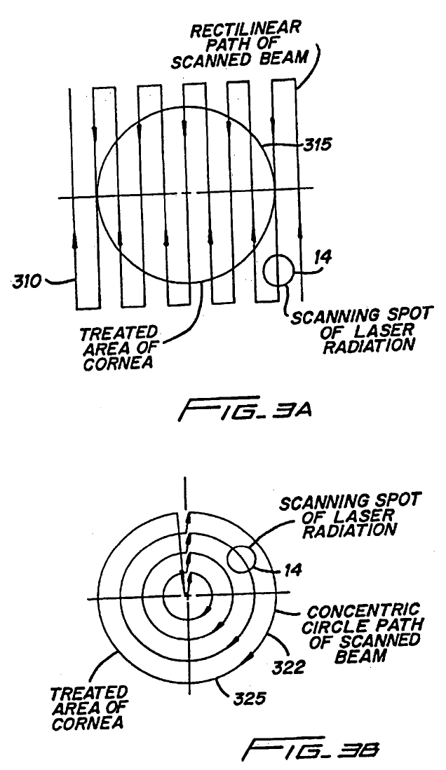

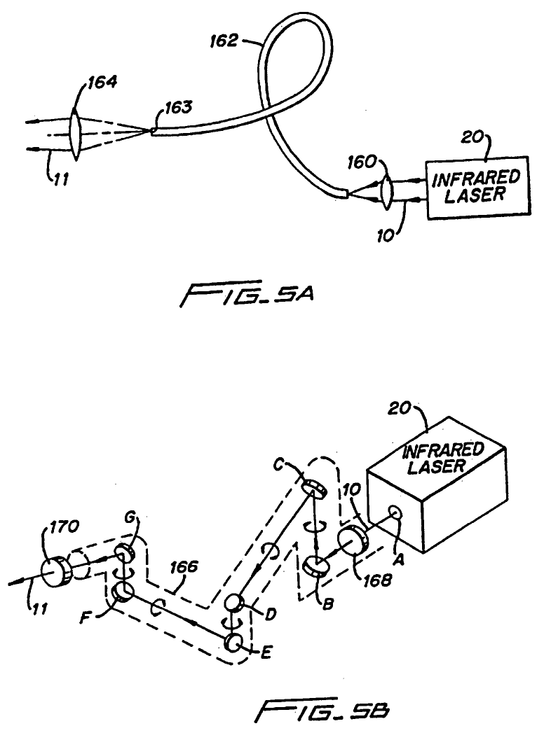

As shown in FIGS. 1 and 2, a surgical apparatus 200 includes an infrared laser source 20 and an optical assembly, including, in sequence, beam transfer optics 30, discussed below in conjunction with FIG. 5, a safety shutter 40, and partially-transmitting mirrors 50 and 60, which cooperate to focus an output beam 10 upon the cornea of a patient's eye 70, for correcting curvature of the cornea or for affecting therapeutic interventions. The laser source 20 is preferably a mid-infrared laser generating short laser pulses, to yield a tissue removal mechanism based on photospallation, discussed below. The laser beam 10 is preferably scanned over a specific central region of the surface of the cornea in a predefined manner, as discussed below in conjunction with FIGS. 3(a) and 3(b), so as to selectively remove tissue at various points within the cornea and thereby cause the curvature of the cornea to change in a predictable and controlled fashion.

According to one feature of the invention, the laser source 20 is preferably a solid state laser, which emits pulsed radiation in the mid-infrared spectral region with an energy density capable of causing ablative decomposition of corneal tissue. As used herein, the term “solid state laser” includes a diode laser. Preferably, the laser emits radiation at a corneal absorption peak, i.e., at a wavelength of approximately 3 microns, such as 2.7 to 3.1 microns, corresponding to the maximal absorption coefficient of water, the main constituent of the corneal tissue. It has been found that absorption of laser energy by the corneal tissue of the eye 70 at such a wavelength results in complete absorption within 1 to 2 microns of tissue depth. As discussed further below, it has been found that the combination of shallow absorption depths and short radiation pulses reduces the undesirable thermal damage of surrounding tissue to insignificant levels.

Photospallation

As previously indicated, according to a feature of the present invention, the surgical technique disclosed herein, whereby corneal tissue is irradiated with short pulses of a scanned mid-infrared laser beam, is based on a concept referred to as photospallation. Generally, photospallation is a photomechanical ablation mechanism which results from the absorption of incident radiation by the corneal tissue. When the corneal tissue absorbs the infrared radiation, a bipolar oscillating shock wave is created, which alternately compresses and stretches the corneal tissue, ejecting the tissue fragments torn apart during the stretching phase. For a more detailed discussion of photospallation, see Jacques, S. L., “Laser-Tissue Interactions: Photochemical, Photothermal, and Photomechanical,” Lasers in General Surgery, 72(3), 531-558 (1992), incorporated by reference herein. Since photospallation is a mechanical ablation process, there is very little heat generated in the adjacent tissue left behind after the ablation.

The laser source 20 may be embodied as a Q-switched Er:YAG laser, which delivers a beam of mid-infrared radiation at or near a wavelength of 2.94 microns. Alternatively, the laser source 20 may be embodied as a Neodemium or Holmium doped laser which is frequency shifted by an optical parametric oscillator (OPO) to emit radiation of approximately 3 microns. Of course, substitution of other alternative laser sources with similar emission characteristics to that of the Er:YAG laser, as they become available, are included within the scope of this invention.

The laser source 20 preferably emits radiation at discrete pulses of less than 50 nanoseconds in duration at a repetition rate of 5 to 100 hertz. The laser pulses should be short enough such that lateral thermal damage of tissue adjacent to the irradiated region is limited to a region smaller than 2 microns wide, consistent with a photospallation process. In addition, the energy in each pulse of the laser 20 is preferably on the order of 5 to 30 mJ. Thus, the incident laser beam 14 will ablate tissue locally and thereby remove microscopic portions of the cornea.

Line-of-Sight

To correlate the eye's reference frame to that of the surgical instrument 200, as shown in FIGS. 1 and 2, it is necessary that the line-of-sight of eye 70 be substantially coincident with the propagation axis of the incident laser beam 14. As used herein, in accordance with customary definition, the term “line-of-sight” or “principal line of vision” refers to the chief ray of the bundle of rays passing through the pupil and reaching the fovea, thus connecting the fovea with the fixation point through the center of the entrance pupil. It will therefore be appreciated that the line-of-sight constitutes an eye metric defined directly by the patient, rather than through some external measurement of the eye and further, that the line-of-sight can be defined without ambiguity for a given eye and is the only axis amenable to objective measurement using cooperative patient fixation.

Since critical vision is by definition centered on the line-of-sight of the eye, irrespective of the direction in which the mechanical axis of symmetry of the eye is pointed, it is generally acknowledged that for best optical performance, the point marking the intersection of the line-of-sight with the cornea establishes the desired center for the optical zone of refractive procedures seeking to restore visual acuity. It is noted that the orientation of the line-of-sight of the eye 70, as shown in FIGS. 1 and 2, may be vertical, horizontal, or intermediate to those extremes as befitting comfortable positioning of the patient for surgery without affecting the validity of the invention.

During preparation for laser surgery on the cornea, the line-of-sight of the eye 70 must be aligned to coincide with the laser beam axis by two-axis lateral-translational adjustments, in a known manner, as directed by the surgeon 55. The surgeon 55 observes the eye 70 through a surgical microscope 80 and judges the degree of centration of the frontal image of the eye 70 with respect to a crosshair or other fixed reference mark indicating, as a result of prior calibration, the location of the axis of beam 14, in a known manner. The axial location of the eye 70 is also judged by the surgeon 55 by virtue of the observed degree of focus of the image of the eye 70 relative to the previously calibrated and fixed object plane of best focus for microscope 80. Directions from the surgeon 55 allow adjustment of the axial position of the cornea of eye 70 to coincide with the plane of best focus.

The required angular orientation of the line-of-sight of eye 70 is preferably established by directing the patient to observe and focus attention, i.e., fixate, on two coaxial illuminated targets (not shown) projected into the eye 70 by a fixation target device 90, which is preferably integrated into the microscope 80. The two targets appear to be located at different axial distances from the eye 70 of the patient and will have been previously calibrated in a known manner. For a description of a suitable calibration technique, see PCT application No. PCT/US94/07908 to Knopp and Yoder. In this manner, when the two targets (not shown) appear superimposed, the axis of the observing eye 70 will be substantially coincident angularly with the axis of the microscope 80 and also with the axis of laser beam 14.

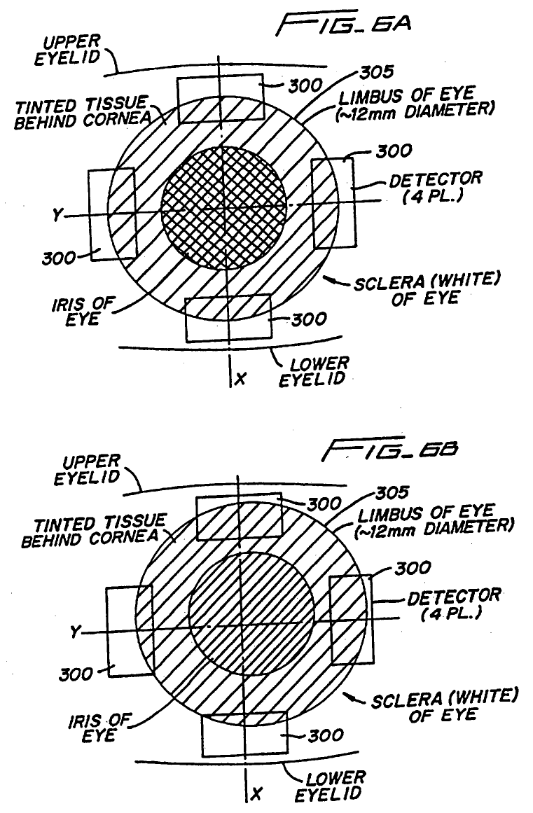

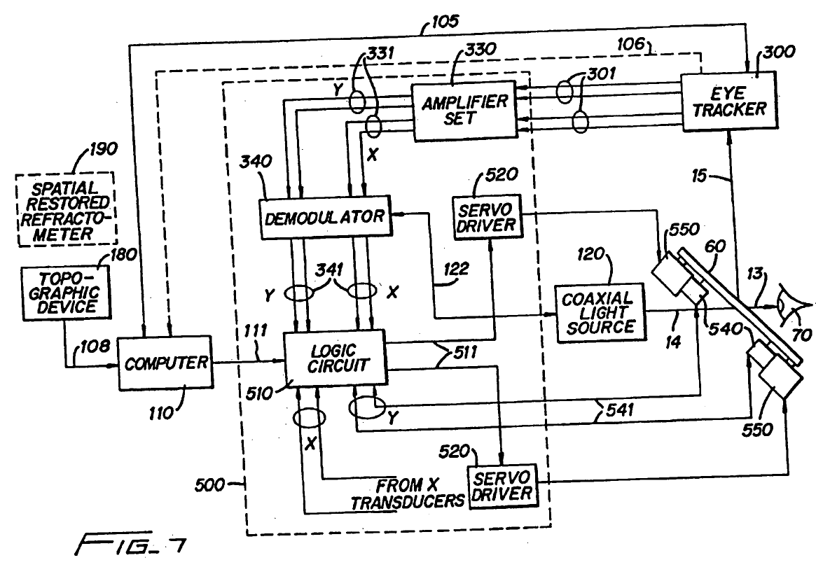

In a preferred embodiment, small lateral motions of the patient's eye 70, i.e., less than 5 mm in either direction, that occur after the initial alignment performed in the manner described above, and throughout surgical treatment, are rendered inconsequential by virtue of the function of a two-dimensional eye tracker 100, discussed further below in conjunction with FIGS. 6 and 7. The eye tracker 100 senses the motion of the eye 70 and provides signals that are proportional to the errors in lateral alignment of the eye 70 relative to the axis of the laser beam 14. The signals generated by eye tracker 100 are converted into commands for small angular tilts of partially-reflecting mirror 60 that compensate for errors in the location of the eye 70. The small angular tilts serve to redirect beam 14 so as to make it coincide with the instantaneous position of the eye 70. The compensation commands are sent from electronics, discussed below in conjunction with FIG. 7, within the eye tracker 100 to mirror 60 by means of one or more data connections, collectively designated 102.

Illumination of the eye 70 to facilitate tracking by the eye tracker 100 is preferably accomplished by means of a coaxial illuminator 120, preferably integrated with the microscope 80, that projects a beam of light 17 at a small angle, on the order of 8 .degree., with respect to the line-of-sight of the microscope 80. According to a feature of the invention, the nature, i.e., the wavelength and temporal modulation frequency, of the tracking beam 17 generated by illuminator 120 is preferably selected to maximize discrimination by the detectors and related electronic circuitry within eye tracker 100 of the tracking beam 17, from ambient room illumination and radiation from laser 20. In this manner, the ambient illumination and laser beam 14 will not possess the same temporal modulation nor spectral characteristics as the tracking beam 17, and will thus be virtually invisible to the tracking detectors.

In addition, as shown in FIG. 1, the surgical system 200 preferably includes a safety shutter 40 which closes automatically if the laser beam 14 fails to follow a prescribed path, if pulse energy-monitoring means provided within laser 20 indicates a malfunction of said laser or if the eye tracker 100 cannot follow the eye motion.

As shown in FIG. 1 and discussed further below, the surgical apparatus 200 preferably includes a video camera 140 that displays a real-time image of the patient's eye on a monitor 150 during pre-operation alignment and during surgical treatment and records the video image on a video recorder 160 for postoperative examination and documentation of the surgical procedure.

As shown in FIG. 1, the computer 110 includes multiple storage and control capabilities. Specifically, the computer 110 communicates and thereby controls the laser source 20 by means of a connection 101. In addition, the computer 110 drives the scanning mirror 50 by means of a connection 103, in accordance with stored scanning patterns and commands input to the computer 110 by the surgeon 55 or an assistant. A connection 104 between the computer 110 and the safety shutter 40 affects maximum safety of the patient, the surgeon, and attending personnel. The computer 110 monitors the operation and status of the eye tracker system 100 by means of a connection 105. Alternately, as shown in FIG. 1, computer 110 can be connected to the eye tracker 100 by means of connection 106 and a separate connection 107 can be provided from computer 110 to mirror 60 so that the computer 110 could directly control the position of the mirror 60. A further alternate configuration would allow the computer 110 to combine the scanning and eye tracking functions together onto a single mirror, such as the mirror 60, thereby removing the need for connection 103.

As discussed further below, the surgical apparatus 200 preferably includes a corneal topography device 180 or a spatially resolved refractometer 190, as shown in FIG. 1. A corneal topography device 180 may be used for evaluating the shape of the corneal tissue to assist in pre-op and post-op measurements of the eyes' shape or curvature. An alternate embodiment would include a spatially resolved refractometer (SRR) 190 to evaluate the refraction of the corneal tissue.

Optical Mirrors

It may be noted from examination of FIGS. 1 and 2 that the partially-reflecting natures of mirrors 50 and 60 play important roles in the proper function of the invention. In the case of mirror 50, laser radiation in beam 12 is reflected while radiation from eye tracker 100 is transmitted. This can be accomplished, for example, through use of what is commonly called a “hot mirror” coating on the surface of mirror 50. This coating is dichroic, in other words, the coating has different reflection and transmission characteristics for light of differing wavelengths. The radiation from laser 20 has a wavelength of approximately 2.9 microns and the mirror 50 should have a high reflectance at that wavelength. The radiation to eye tracker 100 preferably has a wavelength between 0.8 and 1.0 microns for which the coating of mirror 50 should have a high transmittance.

Similarly, the dichroic coating on mirror 60 is preferably selected to have high reflectance at the wavelength of laser 20 and approximately equal transmittance and reflectance at the visible wavelengths used by the surgeon's eye in observing the alignment of the eye with respect to the surgical apparatus and progress of the surgery, at the wavelength of the fixation target 90, and at the wavelength of the coaxial illuminator 120. This is possible since the visible range, the fixation target 90, and the illuminator 120 are adjacent in wavelength and far from the wavelength of laser 20. At both mirrors 50 and 60 the transmitted beams suffer small lateral displacements due to oblique incidence and the finite thickness of the mirror substrates, but these fixed displacements are easily compensated for in the design of the apparatus, as would be apparent to a person of ordinary skill in the art.

In addition, mirror 130, shown between beams 15 and 16 of FIGS. 1 and 2, is also preferably partially transmitting, although not dichroic. The coating on mirror 130 nominally has approximately equal reflectance and transmission characteristics at the wavelengths of the eye tracker light source 120 and throughout a significant portion of the visible spectral region. In this manner, a portion of the energy of beam 15 can be redirected as beam 18 into video camera 140, discussed above. It is understood that a beamsplitting prism, typically in the form of a cemented two-element cube with a partially-reflecting coating on an internal surface can be employed to provide the function of mirror 130.

Scanning Patterns

As previously indicated, the surgical apparatus 200 of FIGS. 1 and 2 preferably provides a computer-controlled scanning motion of the focused laser beam 14 for sequentially irradiating contiguous small areas of the central portion of the cornea of eye 70 with pulses of mid-infrared laser radiation in predefined patterns, such as those illustrated in FIGS. 3(a) and 3(b). In each case, the region to be treated has a diameter of up to 9 mm. The size of the focused spot of laser radiation is preferably on the order of a 0.5 to 2.0 mm circumscribed diameter.

As shown in FIG. 3(a), a rectilinear or raster-scan 310 of the scanning spot of laser beam 14 covers a square area centered on the desired treatment region 315. The laser beam 14 is modulated “off” when the computer 110 predicts that the energy would impinge upon corneal tissue outside the desired treatment region 315. As shown in FIG. 3(b), the laser beam 14 scans in a concentric-circle pattern 322 that is centered on the desired treatment region 325. While the path of the laser beam 14 may be continuous from start to finish, as indicated in the illustrative modes of FIGS. 3(a) and 3(b), an alternative operational mode divides the pattern a list of location coordinates and covers the entire area in a discontinuous fashion in order to minimize residual thermal effects of the area adjacent to the scan path by cumulative irradiation in rapidly sequenced locations of the beam. In this embodiment, the scanner would have random access capability to each location.

In the illustrative modes shown in FIGS. 3(a) and 3(b), or in other continuous or discontinuous scan patterns which would be apparent to persons of ordinary skill in the art, based on the disclosure herein, adjacent scan paths nominally overlap in a controlled manner. In this manner, the entire treatment region 315, 325 is uniformly irradiated with minimal discernible lines of overexposed or underexposed tissue lying between the scans. It is noted that the discontinuous property of the sequence distributes the pulses over the entire area in each time interval which is short compared to the entire sequence, thereby better distributing any residual heat to the entire surface and minimizing the buildup of heat and any temperature rise in any localized area. Once the pattern is defined by the computer 110, the implementation of the delivery can be discontinuously distributed across the corneal surface for maximum surface smoothness and minimum thermal effect.

Scanning of the laser beam over the cornea surface is accomplished by a controlled tilting of the partially-reflecting mirror 50 about two axes so the reflected beam is deviated in an appropriate manner. This scanning motion is imparted to electrically-driven tilting mechanisms attached to mirror 50 under control of computer 110 upon initiation of the surgical treatment.

The velocity of the scan motion is varied at different points within the treatment area 315, 325 in accordance with an algorithm prescribed by the surgeon 55 to cause more or less ablation to take place locally, thereby causing the desired changes in refractive power of the cornea's anterior surface to correct the patient's vision defects. Correction of astigmatic, or cylindrical, errors can be accomplished by driving the scan mirror at different speeds as a function of rotational location about the propagation axis in the pattern. This allows the laser beam 14 to selectively ablate more tissue near one meridian of the corneal surface than near the orthogonal meridian. The nonsymmetric scan motion can be superimposed upon the normal symmetric pattern to simultaneously correct spherical and cylindrical refractive errors.

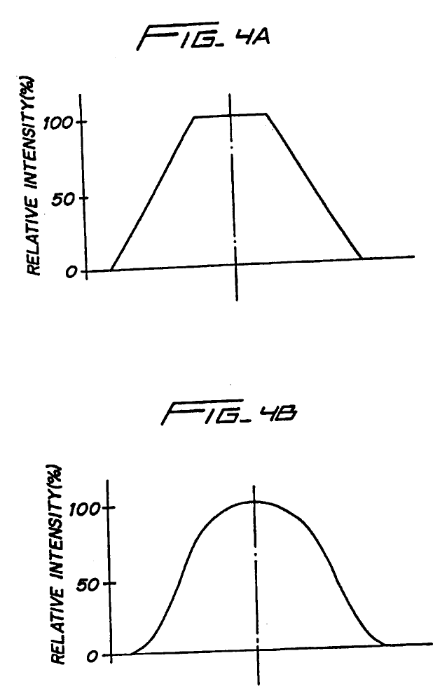

As shown in FIG. 4(a), the intensity profile of the focused laser beam 14 at the corneal surface ideally is contoured as a rotationally-symmetric trapezoid, in order to facilitate uniform irradiation of the treatment region 315, 325. The essentially gaussian profile shown in FIG. 4(b) approximates the idealized intensity profile illustrated in FIG. 4(a). It is noted that for smaller beam diameters, i.e., up to 2 mm, impinging on the corneal surface, the tissue removal profile for excimer ablation approximates a gaussian shape, independent from the beam intensity profile. For intermediate diameters, however, i.e., from 2 to 4 mm, the ablation profile approximates the beam intensity profile of the excimer laser beam. For larger diameters, i.e., from 4 to 7 mm or more, the ablation profile is deeper at the edge than the center compared to the beam intensity profile.

Photospallation is similar to the excimer ablation mechanism described above in that the beam intensity profile is generally not critical to the design or ablation pattern when using a spot size of 2 mm or smaller. Unlike photovaporization, where the tissue ablation mechanism is photothermal, the tissue ablation mechanism for photospallation is photomechanical. Therefore, the ablation pattern depends on the beam diameter, rather than a specific intensity profile. Thus, as a further advantage, since the present invention depends on pulse diameter and is not particularly sensitive to minor variations in the beam intensity profile, laser design issues may be relaxed.

Beam Transfer Optics

As previously indicated, laser beam 10 is transferred to the main portion of the surgical apparatus 200 by means of beam transfer optics 30, shown in greater detail in FIGS. 5(a) and 5(b). It is noted that for the often crowded environment of an operating room, a flexible arrangement, whereby the beam delivery is effectively decoupled from the laser system, is preferred. As shown in FIG. 5(a), the beam transfer optics preferably includes a focusing lens 160 to condense the laser beam 10 into the entrance aperture of a decoupled guided means 162, such as a flexible fiber-optic cable. The fiber-optic cable 162 should preferably be capable of transmitting the intense infrared laser radiation over some distance, i.e., across an operating room, without damage to the fiber-optic cable itself, or significant loss of laser energy.

The fiber-optic cable 162 can be embodied as a single- or multiple-fiber bundle, and comprised of a material that safely transmits the specific wavelength of the laser 20, such as glass, sapphire, or another crystal. It is noted that in the infrared wavelength range, the additional losses associated with the added components required by the decoupled beam transfer optics 30 will generally be quite small. Alternatively, the laser beam can be coupled to the scanner system by means of a flexible hollow waveguide (not shown).

Preferably, the fiber-optic cable 162 connects the laser 20 to the main portion of the surgical apparatus 200 in a manner that permits convenient location of the laser 20 in the vicinity of the surgical apparatus 200, but not necessarily in a specific location. As shown in FIG. 5(a), the laser radiation exiting the output aperture 163 of the fiber cable 162 is captured by a relay lens 164 that forms an image of the output aperture 163. As shown in FIG. 1, this image is then propagated along paths 11, 12, 13 and 14 by means of partially-reflecting mirrors 50 and 60, to position the image at the anterior surface of the cornea of eye 70. The image plane of relay lens 164 is positioned during assembly of the apparatus so as to lie at the plane of best focus of microscope 80. The fiber-optic cable 162 may be embodied as the SapphlRe product, commercially available from Saphikon, Inc., or in accordance with the teachings of U.S. Pat. No. 5,349,590.

An alternate embodiment of the beam transfer optics 30 is shown in FIG. 5(b). The alternate arrangement of FIG. 5(b) replaces the fiber-optic cable 162 of FIG. 5(a) with a flexible articulated arm 166. The flexibility of the articulated arm 166, by rotation about axes B-C, C-D, D-E, E-F, and/or F-G, allows convenient location of the laser source 20 with respect to the main portion of the surgical apparatus 200, again without requiring the laser source 20 to occupy a specific location. Condensing and relaying of the laser radiation at input and output apertures of the articulated arm are accomplished by means of lenses 168 and 170 in a manner substantially as described for the corresponding optical components in FIG. 5(a). The articulated arm 166 may be embodied as the Light Guiding Arm, commercially available from Dantec measurements Technology, or in accordance with the teachings of U.S. Pat. No. 4,896,015.

Another alternate embodiment for the beam delivery system would place the laser on the arm of the surgical microscope in a fixed location with respect to the main portion of the surgical apparatus 200. Such an arrangement would require certain rigid relay means to transport the radiation, which may require greater care in optical alignment, while imposing additional packaging constraints. For these and other reasons, the decoupled means of FIG. 5(a) and FIG. 5(b) are preferred.

Eye Tracker

The importance of proper centration of the treatment is generally recognized as an important factor for all PRK procedures. Misalignments of the eye 70 during the procedure are known to result in irregular astigmatism, glare phenomena, and decreased visual acuity and contrast sensitivity. Thus, as previously indicated, the surgical apparatus 200 preferably includes an eye tracker 100 which senses the motion of the eye 70 and provides signals that are proportional to the errors in lateral alignment of the eye 70 relative to the axis of the laser beam 14. An illustrative prior art eye tracking technique is disclosed in PCT Application No. PCT/US94/02007 to Knopp, et al, incorporated by reference herein.

The eye tracker 100 senses lateral movement of the patient's eye 70 by forming an optical image of a significant feature of the eye on an array 300 of detectors preferably arranged in the manner depicted in FIG. 6. The eye feature imaged by the eye tracker 100 is the approximately circular intersection 305 of the transparent cornea with the translucent and white-colored sclera constituting a structural member of the eyeball 70.

The intersection 305 is commonly known as the limbus of the eye 70. The limbus is approximately 12 mm in diameter in the human eye and is easily seen by virtue of its circular geometric contour and the inherent coloration of underlying ocular tissue seen through the transparent cornea as compared with the white sclera. In frontal view, transition at the limbus from the colored or tinted circular area and the white sclera offers photometric contrast in an axi-symmetric feature of the eye 70 that lends itself to tracking by the means described here. In a preferred embodiment, the contrast can be further enhanced by using red or near infrared filters in front of the detectors to make blue and green pupils appear as dark as brown pupils to the detector array 300.

When the limbus feature of the eye 70 is perfectly centered with respect to the axis of laser beam 14, the image of the limbus formed by lens 320 is centered on the detector array 300, as shown in FIG. 6(a). Under this centered condition, the four detectors comprising the array 300 each receive essentially equal amounts of energy from the image of the limbus 305 and, with the assistance of associated electronic means (not shown), create a null signal that is transmitted to tracking mirror 60 via connection 102 which serves to hold the mirror 60 stationary in its current position.

When the eye 70 is not perfectly centered with respect to the axis of the laser beam 14, however, the image of the limbus 305 formed at the detector array 300 is more or less decentered, as indicated schematically in FIG. 6(b). Under such a decentered condition, unequal amounts of light energy are deposited on the four detector elements comprising the array 300 and error signals proportional to the lateral displacement are created by the aforementioned associated electronics. These error signals are transmitted to the drive mechanism of mirror 60 causing the mirror 60 to deflect as required to return the image to its centered position.

Accordingly, the function of the eye tracker 100 is to maintain a centered condition between the axis of beam 14 and the cornea of eye 70. In this manner, the laser radiation delivered through beam 14 is applied to the cornea as if the eye had not moved from its nominal centered position. In addition, in order to allow for real-time tracking, the above-described tracking algorithm is preferably performed at least once for each interpulse duration. Thus, in the illustrative embodiment, where each pulse has a duration of less than 50 nanoseconds, at a repetition of 100 Hertz, there will be 10 milliseconds between pulses and the eye tracking response time is preferably less than 10 milliseconds.

It is understood that the scanning algorithm which is applied to mirror 50, in the manner described above, and the eye tracking function which is applied to mirror 60, could be combined and applied onto a single mirror, such as the mirror 60. In this embodiment, the mirror 50 would be a fixed mirror/beam splitter. This configuration could reduce hardware cost but would complicate the logical operation of the system and could increase the angular range requirements of the single mirror 60. Using two separate mirrors reduces the range requirements for each mirror and simplifies the design, manufacture, and testing of the separate scanning and eye tracking functions.

As previously indicated, frontal illumination of the eye 70, which is essential to proper functioning of the eye tracker 100, is provided by the coaxial illuminator 120 which may be integrated with the microscope 80. The illuminator 120 projects a tracking beam 17 onto the eye 70. Light reflected and scattered differentially by the cornea and underlying tissue and the adjacent sclera at the limbus 305 constitutes the object imaged by lens 320 at an appropriate magnification onto the detector array 300.

In a preferred embodiment of the invention, the wavelength of the illuminator beam 17 is chosen in the near infrared range of wavelength at approximately 0.8 to 1.0 microns. The sensitivity of the human eye is very low at those wavelengths so the portion of the beam 17 reflected by the cornea surface back into the microscope 80 will be so small as to not affect observation of the patient's eye by the surgeon 55 through the microscope 80. In addition, because of its low visibility to the eye 70, the near infrared frontal illumination also will not interfere with fixation of the eye by the patient upon the visible light sources, or targets, located within fixation target device 90.

Further, the intensity of the light source within illuminator 120 can be modulated at some convenient temporal frequency so as to further facilitate discrimination from unmodulated room ambient illumination or laser beam 14 by appropriate synchronous filtering within the electronics associated with the detectors of array 300. The detectors of the array 300 are not sensitive to the infrared radiation from laser source 20, so will not respond to laser beam 14 during operation of the eye tracker 100.

By virtue of the near angular coincidence of tracking beam 17 and laser beam 14, the specular reflection of tracking beam 17 from the cornea occurs near the center of the cornea and well inside the limbus 305. This reflection will therefore not interfere with the eye motion sensing function of the eye tracker system since it will not be imaged by lens 320 onto the detectors comprising array 300. It has been found that the use of a temporally modulated infrared light source, and the favorable choice of angular incidence of the beam 17 of illumination from said source onto the cornea of eye 70 constitute distinct improvements in the state of the art as represented by PCT Application No. WO 94/02007.

FIG. 7 shows, in schematic form, one embodiment of a servo system 500 used to drive the tracking mirror 60, along with the associated input signals from tracking detectors 300 contained within eye tracker 100 and related controls. In a preferred embodiment of the invention, the four detectors, collectively labeled 300 in FIG. 2, each comprise a single element PIN silicon photodetector, although dual-element detectors may alternately be selected, based upon specific functional requirements of the instrument.

Voltage signals 301 received from the detectors are subsequently fed into amplifier set 330, with the amplified signals 331 channeled directly into a demodulator 340. This demodulator is temporally synchronized, as indicated by control 122, with the tracking light source 120 used to illuminate the eye to ensure that only light of the appropriate frequency is selected for the tracking signals. As previously indicated, this synchronization constitutes a means for temporal differentiation of reflected light used for tracking, thus further enhancing signal levels over ambient light background. The gated signals 341 emerging from the demodulator are then fed into the logic circuit 510.

The logic circuit 510 comprises a key element of the servo subsystem, and serves as the central switchboard of the closed tracking feedback loop. The logic circuit converts the amplified and demodulated signals from the detectors of the array 300, corresponding to target position, into commands for controlling the tracking element, in this case, the tracking mirror 60. It is to be understood that diametrically opposing pairs of detectors produce varying electrical outputs as the image of the limbus 305 moves with respect to the X and Y axes, as indicated in FIG. 6.

The arithmetic difference between signals from each pair of opposing detectors is substantially proportional to the displacement of the image from the centered or null position in the corresponding axis. The signal differences produced within logic circuit 510 and further processed by the circuit 510 constitute mirror displacement commands indicated by controls 511. These displacement commands are relayed to the servo drivers 520 which, in turn, activate actuators 550 which are mechanically linked to mirror 60, thus causing the mirror 60 to pivot about its axes. In this manner, the angular orientation of the mirror 60 may be modified as required to pursue the target motion in two dimensions.

Transducers 540 are also mechanically connected to mirror 60 to provide feedback to logic circuit 510 via connections 541. The transducers 540 generally are comprised of position-sensing elements which, in a preferred embodiment, are simple, readily-available components. The transducers 540 allow stabilization of the motion of the tracking element, in this case mirror 60, referenced to a pre-selected default position. In addition, the transducers 540 sense when the tracking mirror 60 is at the end of its range and will no longer track the motion. This enables the computer 110 to stop the laser source 20, or to close shutter 40, when the tracker is no longer able to follow the eye motion.

In the preferred embodiment, the reference position of the mirror 60 corresponds to alignment of the patient's line-of-sight with the optical axis of the instrument, as previously discussed. This reference position can be selected by the computer 110, when the surgeon 55 indicates that the patient is aligned. Note that the collection of signals shown in FIG. 7, designated 301, 521, and 541 from the eye tracker 300 to the tracking mirror 60 were denoted collectively as connection 102 in FIG. 1. It is noted that for visual clarity, FIG. 7 illustrates only two of each of the four servo drivers 520, transducers 540 and actuators 550 that would be included in the illustrative servo system.

Like most servo systems, the system shown in FIG. 7 is an off-null measurement system based on returning the errors signals to zero. There may be alternative implementations of a servo control system other than the one depicted in FIG. 7 which would still allow the accurate measurement and/or control of eye displacements at sufficiently high rates. Such alternative servo systems are therefore included within the scope of the present invention.

Topographic Measurements

As previously indicated, a corneal topography device 180 may be used to assist in pre-op and post-op measurements of the eyes' shape or curvature. Any commercially available topographic instrument may be used for this purpose as long as it is modified to include reference targets for fixation as utilized by the present invention. An alternate embodiment would include in this location a Spatially Resolved Refractometer (SRR) 190 to measure true refraction across the cornea.

The ability to establish a common reference frame between different ophthalmic instruments is of further importance in consideration of the desirability of integrating the method of corneal surgery that is the subject of the invention with independent refractive and/or topographic measurements of the cornea. It is generally recognized that accurate measurement and determination of the refractive status of the eye is desirable for a successful outcome of any refractive surgical procedure.

Corneal topographic devices, such as those manufactured by EyeSys and Computed Anatomy, have had some utility in providing evaluation of pre- and post-operative shape of the cornea. Other instruments that have recently become available, such as the OrbScan product by Orbtek, Inc., may provide information about the local shape of the cornea which can be highly useful for optimizing the correction of certain types of refractive errors, such as astigmatism. For any of these instruments to be effective, however, it must be compatible with repeated measurements being referenced to the same location in the eye. This aspect can be provided by an eye tracking or fixation technique, in the manner described above, that is unique to a patient and not to an instrument. Inclusion of such an alignment feature may also allow intraoperative measurement of corneal topography which could be used as an active feedback during the procedure for the purpose of enhancing the precision of surgery and eliminating undesirable variables affecting predictability. Prior art as described by U.S. Pat. No. 5,350,374 to Smith shows the possibility of integrating an active feedback control loop based on a particular type of topographic instrument with a corneal surgery procedure.

In various embodiments, the present invention also seeks to include topographic feedback that is compatible with any number of available corneal measurement devices thus incorporating many of the advantageous features of the prior art devices, but enlarging their scope to include PRK surgery with a mid-infrared laser using a scanning beam delivery system.

An alternative to the shape mapping of these topography devices is the refraction mapping device and method called Spatially Resolved Refractometer (SRR). For a detailed discussion of SRR, see Webb, R. H., Murray Penny, C., Thompson, K. P., “Measurement of Ocular Local Wavefront Distortion with a Spatially Resolved Refractometer,” Applied Optics, 31, 19, 3678-3686 (1992). The SRR device measures the refraction at each point on the cornea over the pupil by having a patient align two fixation sources through a small pinhole. This pinhole is translated across the cornea to map each point of the cornea with a separate refraction measurement. Since the purpose of PRK is to correct the refractive error of a patient, the SRR map is the ideal input for correction by the PRK system, providing an improvement over the refraction measured in a refracting lane, as well as the power map from a topography system. This preoperative input data may be used to help define the ablation profile and pattern. Alternatively, SRR may to used to map the eye during a procedure.

A mid-IR laser source is disclosed with parameters selected to yield a beam with properties matched to optimal tissue removal based on a photospallation mechanism. Optimally, the laser beam comprises a series of discrete pulses of less than 25 ns in duration, each with energy of greater than 1 mJ emitted at repetition rates of at least 10 Hz, but scalable to over 50 Hz. High repetition rate is required to minimize the duration of the medical procedure while allowing small spot sizes with better overlap parameters to be utilized for improved surgical outcomes. The critical nature of the pulse duration is related to the threshold for the photospallation process, which is expected to be lower as the pulse duration decreases thus allowing for lower energy densities (or, fluences) to be utilized to affect ablation. Generally, the lower the energy density, the less likely it is that thermal damage to tissue surrounding the ablation site will occur. This, in turn, is an important factor in producing highly localized ablation with clinical results similar to what is obtained currently with UV radiation.

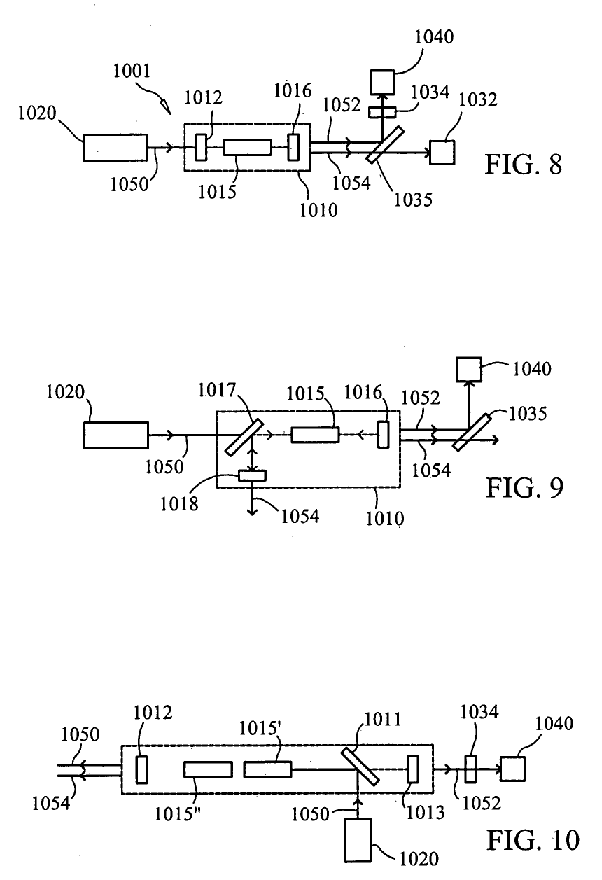

As shown in FIG. 8, a mid-infrared laser source 1001 preferably includes a neodymium-doped laser source pump 1020, generating a pump beam 1050 comprised of short laser pulses (preferably less than 30 ns) at or around 1 micron, which radiation is down-converted to the mid-IR wavelength range through an Optical Parametric Oscillator (OPO) 1010. The OPO 1010 is shown to include mirrors 1012, 1016 and a nonlinear crystal 1015. The effect of the nonlinear crystal 1015 on the laser pulses results in two beams, in a known manner. Specifically, the output of the OPO comprises an idler beam 1052 and a signal beam 1054. For a detailed description of the operation of one particular OPO, see U.S. Pat. No. 5,181,211, incorporated by reference herein.

For refractive surgery, the desired wavelengths are those of the idler beam 1052, which in the preferred embodiment fall in the range between 2.89 and 2.98 microns. In the example of a Nd:YAG pump beam at 1.064 microns, the corresponding wavelength of the signal beam 1054 is between 1.68 and 1.66 microns. It is to be understood, however, that while a wavelength near the 2.94 microns water absorption peak is preferred, especially for PRK applications, idler wavelengths anywhere in the range of approximately 2.75 to just over 3.0 microns fall within the scope of the invention, with the specific wavelength chosen to match the needs of the surgical application.

The idler beam 1052 is reflected from dichroic beam splitter 1035 and is subsequently directed to beam transfer optics 1040, which, in a preferred embodiment may include imaging and scanner means to allow selective removal of tissue at various points on the cornea, thereby causing the cornea to change in a predictable and controlled manner. Such means were disclosed in our co-pending parent application, U.S. Ser. No. 08/549,385, incorporated herein by reference, and are not considered critical to the present invention. The signal beam 1054 is transmitted through the beam splitter 1035 to a beam dump 1032. Further attenuation of the residual signal beam 1054 may be provided by additional reflectors collectively represented as attenuator 1034 which may be placed in the path of the idler beam 1052 to prevent any coupling of the signal wavelengths from the signal beam 1054 into the delivery system 1040.

In the embodiment of FIG. 8, the coatings and positioning of the mirrors 1012, the crystal 1015 and the mirror 1016 in the OPO cavity 10 are chosen to comprise a singly resonant oscillator (SRO) configuration optimized for producing the idler wavelengths and with the added feature of using backreflection of the unconverted portion of the pump beam 1050 into the crystal for further processing. Thus, mirror 1012 is coated for high transmission of wavelengths between 1.0 and 1.1 microns and high reflection of the idler wavelengths between 2.8 and 3.0 microns. Mirror 1016 is coated to have partial reflectance for wavelengths between 2.8 and 3.0 microns and high transmission at the 1.65 to 1.7 microns wavelengths characteristic of the signal beam 1054. The signal beam 1054 thus passes through the oscillator cavity without reflection, while the idler beam 1052 is resonated to assure maximum output at the mid-IR wavelengths. Preferably, mirror 1016 is also coated for high reflectance at the pump wavelengths between 1.0 and 1.1 microns. It is not, however, essential to provide this last high reflectance but such reflection may be advantageous for more efficient operation of the device by lowering the energy threshold for the parametric process.

An alternative configuration to the SRO is that of a Doubly Resonant Oscillation (DRO), where both the idler and signal waves are resonated. In general, a DRO is known to have a lower oscillation threshold but has the drawback of more complicated mirror coatings, and somewhat more difficult alignment procedures. Nonetheless, while an SRO is preferred due to greater simplicity and lower cost of components, DRO configurations are considered an alternative embodiment for cases where a substantially reduced oscillation threshold presents an advantage. It should be noted that while DRO outputs are known to be less stable than those of an SRO, this is not an issue for this present application where only pump beams comprising a multiplicity of longitudinal modes are utilized. A DRO is therefore an acceptable variation in all the OPO configurations discussed herein.

The surfaces of mirrors 1012 and 1016 may be flat, concave or convex, as would be apparent to a person of ordinary skill. In the preferred embodiment, flat surfaces are advantageous for converting multimode pump radiation, because mode matching would then be dominated by the pump beam 1050, rather than the OPO cavity. Efficiency reduction due to higher order transverse modes is not as severe in this case. Since the resonator mode of a plane parallel OPO consists of a beam of parallel light, a lens to focus the pump beam is also not required, thereby resulting in further simplification of the overall OPO laser design. Alternatives using concave-convex surfaces are possible, but are somewhat more complex to align, as a lens would then have to be provided to match the waist of the pump to the small waist of the OPO resonator mode, further requiring a single transverse-mode pump to assure high OPO efficiency. Mode matching is an important consideration in this type of configuration since any mode mismatch will cause a reduction in gain for optical parametric oscillation and a subsequent increase in threshold. In the preferred embodiment, a less complex and cheaper pump laser would provide a multi-mode beam, with the limits on allowed divergence dictated by the needs of the delivery system rather than the OPO.

The pump laser 1020 consists generally of a neodymium-doped laser rod, such as Nd:YAG, pumped by either flashlamps or diode arrays. Both flashlamp and diode pumped lasers of the required energy, peak power and repetition rate are well known and commercially available. Other appropriate laser media include crystals such as Nd:YLF, Nd:glass and Nd:YAlO₃, all of which provide the fundamental radiation at wavelengths falling in the range covered by the present application.

The crystal 1015 preferably comprises a nonlinear material having high nonlinear coefficient, reasonably wide angular and temperature bandwidths, high damage threshold and minimal absorption at the idler or signal wavelengths. Ideally, a crystal that can be phase-matched non-critically would be preferred, since that would result in the largest possible walk-off angles allowing laser beams with even poor beam quality to be readily converted in long crystals. In a non-critical phase matching (NCPM) arrangement, the crystal is oriented such that phase matching is achieved along a propagation direction parallel to one of the crystal's principal axes (X, Y, or Z). In practice, it may not be possible with currently available materials and lasers to fulfill this criteria for a given application. Alternatively, a crystal with critical phase matching (CPM) may be acceptable as long as the walk-off angles and angular bandwidths are sufficiently high to allow efficient conversion of beams that are not necessarily single transverse mode. We have determined that the crystal known as Potassium Titanyl Phosphate (KTiOPO₄ or “KTP”) is capable of fulfilling the requirements of this application, even though KTP could not be non-critically phase matched with the idler wavelengths of choice generated under pumping with a 1.06 micron wavelength laser. The KTP crystal is also known to exhibit some absorption at or near 3 microns, usually attributed to the presence of residual OH.sup.—radicals inherent to the growth process. Such absorption, if overly large, would seem to hinder the use of KTP for higher repetition rate applications.

We have determined, however, that under the right conditions, KTP is suitable as an OPO crystal for the corneal sculpting application, even with the level of absorption present with current material growth capability. As discussed below, this has been achieved by the fortuitous combination of KTP's large temperature bandwidth and modest energy output and average power requirements of the surgical applications contemplated. With a crystal cut for Type-II phase matching, internal angles of 68 to 70 degrees would provide the required wavelengths for the idler when pumped by a 1.064 microns Nd:YAG laser, based on known material parameters for x-cut material. These angles may be sufficiently close enough to 90 .degree. to provide acceptance angles large enough to accommodate multi-mode pump beams with divergence exceeding many times the diffraction limit, if required. It is to be understood, however, that a judicious selection of components is necessary to achieve the operational conditions required of the surgical laser instrument, especially when the criterion of a compact, simple device consistent with portability in the field is factored in. Measured against the stringent parameters imposed by, for example, the corneal sculpting application, the particular combinations of various OPO elements and parameters using available materials and optics in the simple optical arrangement depicted in FIG. 8 was not apriori obvious.

Accordingly, in one key aspect of this invention, a KTP crystal of sufficient length must be selected to allow efficient conversion of the 1 micron radiation. In a preferred embodiment, crystal lengths of at least 20 mm but potentially as long as 30 mm are appropriate, based on trade-offs of the walk-off angles that are realizable in a 68 to 70 .degree. Type-II CPM configuration for the x-cut crystal and estimates of the OPO gain required to produce idler output energy levels in the desired 5 to 30 mJ range. At this orientation, the acceptance angle for KTP is on the order of 5 cm-mrad, which is still large enough to accommodate the multi-mode pump preferred for the present application.

It is also to be understood that the specific wavelength of the output beam 1052 can be altered by rotating the crystal with respect to the principal axes. This is a potentially useful feature in the surgical context since absorption properties may differ among different types of tissues and, for example, even within the same tissue, as a function of temperature. Hence, a slight variation of wavelength could allow matching to the optimal absorption desired for a given procedure, thus enlarging the scope and utility of the OPO laser source. The limitation on the wavelength range that can be so obtained is determined by the relative sizes of the pump beam and the crystal aperture. Based on known parameters of KTP and the crystal sizes that are readily available, a wavelength range extending from 2.75 to just over 3 microns can all be covered with the present configuration, using any one of several commercially available neodymium-doped pump lasers.

Yet another important aspect of the invention relates to utilization of sufficiently short pump laser pulses such that OPO thresholds may be reached even with an unfocused pump beam arrangement. By eliminating the need for focusing the beam into the crystal, multimode or unstable resonator pump beam spatial distributions may be utilized, which has the advantage of significantly relaxing the requirements for a pump laser while alleviating difficulties associated with the OPO mode matching. In the preferred embodiment, pump pulse durations (FWHM) between 5 ns and 12 ns were found to be acceptable, producing efficient conversion to the idler's wavelengths of over 10% even for a multimode pump beam with divergence greater than 8 times the diffraction limit.

In another feature of the invention, bare crystal faces (i.e., non-anti-reflection (AR) coated) could be used to alleviate risk of damage associated with deficiencies of current coating technologies, whereby residual absorption near the 3 micron wavelength of choice can lower damage thresholds to impractical levels especially when short-duration pulses are utilized. Should high quality, 3 micron coatings become available for KTP, they could be used to advantage as this would lower the OPO losses and allow further reduction in the threshold for parametric oscillation for the same slope efficiency. It should be pointed out, however, that for optimal performance and damage-free operation, the threshold should be such that the desired idler energy output is achieved with an input energy of no more than 3-4 times the threshold. By AR-coating the crystal, the reflectivity of the output coupler can be decreased, thereby dropping the circulating 2.9 microns power for the same output energy.

In the example quoted above, it was determined that with a bare crystal, damage to either the crystal or the optics could be avoided even with input pump energies in excess of 250 mJ for a 10 Hz beam, using all standard optics. Again, the ability to use unfocused beams with diameters on the order of 1 to 5 mm is considered a critical aspect in achieving this performance. To further suppress the potential for damage, especially on the input mirror which is subjected to the full pump power, other arrangements can be employed whereby the pump beam is not coupled through the same 0 .degree. input mirror that must also provide high reflection at 3 microns. There are indications that reflecting the 3 micron idler beam at 45 .degree. instead can increase the damage threshold when the best available 1 micron coatings are used.

Referring now to FIG. 9, an alternative embodiment is illustrated, in which an “L-shaped” cavity is employed using the three mirrors indicated as 1016, 1017 and 1018 to provide some separation between the path of the pump beam 1050 and the idler beam 1052. Thus, the pump is coupled through a 45 .degree. mirror 1017 which is coated to also provide high reflection (at 45 .degree.) at the idler wavelengths. Mirror 1018 is also coated to reflect the idler beam 1052, but it is not subjected to the high power pump beam 1050. The idler beam 1052 is then coupled out through mirror 1016, which is partially reflecting at the wavelength of the idler beam 1052. Again, as in FIG. 8, mirror 1016 is preferably coated to provide back reflection of the pump beam 1050, to lower the threshold for the parametric process. The advantage of this “L” cavity is that the fluence on the input mirror is reduced due to the 45 .degree. angle of incidence. Since this mirror 1017 is typically the first component to damage, lower fluence translates into reduced probability of damage to the OPO at a given level of energy output.

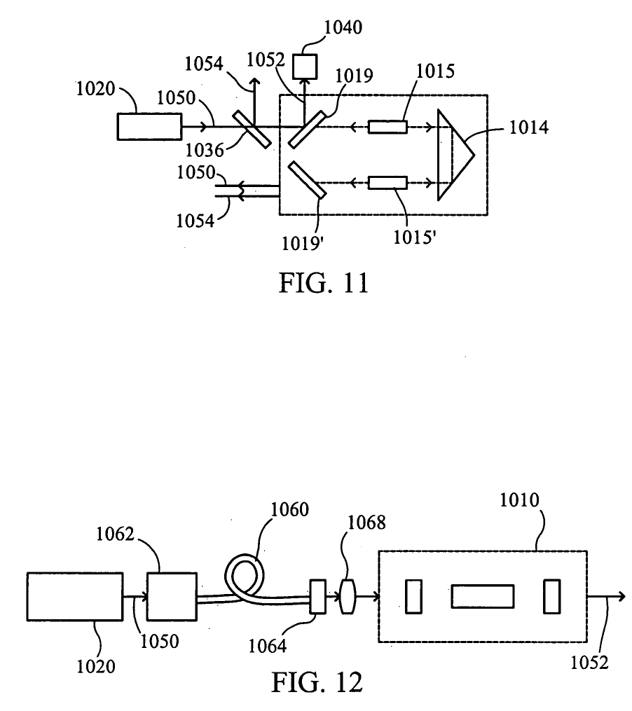

It is to be noted that in the embodiments of both FIGS. 8 and 9, the OPO axis must be slightly offset from the pump axis to prevent feedback into the pump laser 1020. As an alternative, an isolator can be used between the pump laser and the OPO, although that would result in additional cost to the system. FIGS. 10 and 11 represent two alternative configurations that have no pump feedback as they rely on single-pass pumping. Thus, to increase conversion and reduce threshold, instead of back reflection of the pump into the same crystal, two OPO crystals are used in tandem. FIG. 10 shows an arrangement whereby the pump beam 1050 is coupled into the OPO cavity through a 45 .degree. mirror 1011 that is coated for high reflection at the pump wavelengths and high transmission at the idler wavelengths. The pump beam passes through two nonlinear crystals 1015′ and 1015″ and is then transmitted out of the cavity through a mirror 1012 that is coated for high transmission at the pump wavelength and high reflection at the 3.0 microns wavelength range of the idler beam 1052. The idler beam 1052 is coupled out of the cavity through a mirror 1013 that is coated to partially reflect the idler wavelengths with the reflectivity selected to optimize the output from the cavity. In this singly resonant oscillator (SRO), each of the mirrors 1011, 1012 and 1013 are coated to transmit the signal wavelength so that only the idler wavelength is resonant. An alternative arrangement would utilize a DRO which requires reflective coatings at the signal wavelength as well, and possibly also an additional beam splitter and/or other optics. The threshold would then be lower, but at a cost of increased complexity to the optics and in alignment procedures.

FIG. 11 depicts a so-called “ring” configuration, where a prism 1014 provides total internal reflection (TIR) of the beams in the cavity to thus pump two OPO crystals, marked again as 1015 and 1015′ in a single pass arrangement. Two 45 . degree. mirrors 1019 and 1019′ are coated to provide high transmission at the pump and signal wavelengths. Mirror 1019′ is also coated to reflect the idler wavelength, while mirror 1019 is partially reflective at 3 microns to outcouple the idler beam 1052. As FIG. 12 shows, the residual pump beam 1050 is now exiting the OPO cavity via mirror 1019′, thus posing no feed-back problems. Also, since most of the signal beam 1054 is transmitted out of the cavity also through mirror 1019′, there is less of a requirement for further attenuation of the signal in the path of the idler beam 1052. While attractive on these last two counts, the configuration of FIG. 11 is optically more complex, requiring additional elements as compared to the simple arrangement of FIG. 8.

FIG. 12 depicts substantially an alternative novel arrangement using a wave guide means 1060 to couple the pump radiation into the OPO. In a preferred embodiment, the waveguide means comprises a hollow waveguide, a fiber or a fiber bundle. The advantages of using fiber delivery over an air path, fixed beam delivery system for a medical laser system are well known. They include easier alignment of the beam to the surgical site, more flexible adjustment of radiation, delivery angle and location, homogenization (or spatial smoothing) of a multimode beam and the ability to deliver radiation to internal locations not otherwise accessible. However, while fibers for transmitting 1 micron radiation are well developed with damage threshold that can withstand 100's of millijoules of short-pulse radiation, there are not similar fibers currently available to transmit short-pulse, 3 micron radiation. It would therefore be beneficial, if the higher power 1 micron pump beam could be transmitted over a fiber, allowing placement of the OPO in close proximity to the surgical microscope. Most of the advantages of a fiber delivery system would carry over when it is the pump light coupling through a fiber, with the exception of accessing internal locations. In particular, homogenization of the pump beam would result in a smoother profile for the output mid-IR beam, a highly desirable attribute in corneal ablation.

In the embodiment of FIG. 12, the pump beam 1050 is coupled through lenses 1062 into a fiber 1060, which may, in an alternative embodiment consist of a polarization preserving fiber bundle or a hollow metal waveguide. A bundle may be suitable for accepting and transmitting a divergent pump beam 1050 efficiently while allowing for collection and recollimation of light at the distal end through standard optical means 1064. A lens 1068, is shown as imaging the pump light into the OPO. In a preferred embodiment, the lens provides 1:1 imaging, assuming a 6 mm diameter bundle, to preserve the characteristics of the unfocused pump beam arrangement. Other aspect ratios are feasible, depending on the characteristics of available pump beams and fiber numerical apertures. In the preferred embodiment, the bundle may consist of a number of polarization preserving single mode fibers, as required to allow phase matching in the OPO crystal. Using this method, the damage limit of each fiber and the divergence of the beam(s) exiting the fiber(s) must be addressed, as would be apparent to a person of ordinary skill. In the case of the hollow metal waveguide, there are indications that polarization may be preserved and that a waveguide with approximately 1 mm diameter can deliver well over 100 mJ short pulse light at 1 microns wavelength. Such optical means as needed to correct residual depolarization of the pump light exiting waveguide 1060, may be included as part of optical element 1064 in the schematic of FIG. 12. For simplicity, only the simple OPO configuration of FIG. 8 is illustrated in FIG. 12, but it is to be understood that any of the alternative OPO embodiments of FIGS. 9 through 1011 can be used as the OPO element 1010 in FIG. 12.

It is to be noted that absorption in the KTP crystal of choice at or near 3 microns can limit scaling the repetition frequency of the OPO laser source of any of the configurations depicted above. Thus, absorption levels of 8-10% through the length of the crystal were found to be acceptable for the below 0.5 W average power OPO outputs considered this far, a result attributed to the unusually wide temperature bandwidth of KTP. However, it is recognized that to scale the repetition rate of the OPO to beyond 40-50 Hz may require some progress in the material area, whereby growth can be done under altered conditions that do not favor formation of the absorbing OH.sup.—ions. Such advances are currently contemplated, and should they be realized, would allow scaling the repetition rate to beyond the 50 Hz level. Additional scaling of the repetition frequency to the 100 Hz level can also be provided, for example, by interlacing the outputs of two OPOs, pumped by a single laser beam. These, as well as other arrangements utilizing a multiplicity of crystals, fall under the domain of the present invention.

Alternative KTP isomorphs such as KTA and RTA are also recognized as candidates for a mid-IR OPO laser using any one of the configurations specified above, given that they have similar properties to KTP. The selection of a particular crystal thus depends on a combination of characteristics, primarily related to favorable phase matching and minimal absorption at the wavelengths of choice for the present application.

Finally, there are a number of alternative OPO technologies that should they be developed in the near future could be used to advantage in the surgical OPO laser disclosed herein. Such improvements include use of a periodically-poled (PP) KTP which may provide drastically lower thresholds due to high nonlinearities. Output energies from a PP KTP are currently limited to less than 1 mJ due to small (<1 mm) apertures, but larger PP KTP crystals may become available through evolving technologies such as fusion bonding. Furthermore, in a periodically-poled form, LiNbO₃ pumped at 1 microns may also be a candidate crystal for producing the requisite 2.9-3.0 microns wavelengths under quasi phase-matching conditions which effectively simulate NCPM. Apertures are again limited to less than a mm, but future developments may result in larger PP crystals becoming available in the not too-distant future. Of course, absorption in LiNbO₃ at 3 micron remains a problem which will have to be addressed especially for higher repetitious rates.

We also note that utilization of a pump laser source with output wavelengths in the 0.85 to 0.9 microns range represents another alternative OPO configuration. With this pump wavelength, it is possible to non-critically phase-match KTP (x-cut), which would be highly beneficial to the surgical applications contemplated. Unfortunately, pump lasers providing such near-infrared radiation are not yet available as compact low cost, commercial lasers. Candidates include lamp-pumped Ti:sapphire and Cr:LiSAF, neither of which is readily available with the required energy (greater than 100 mJ), pulse duration (less than 25 ns), and repetition rate (greater than 10 Hz) capability. These or similar lasers may however be developed in the future and are thus included within the scope of this invention.

It is to be understood that the embodiments and variations shown and described herein are merely illustrative of the principles of this invention and that various modifications may be implemented by those skilled in the art without departing from the scope and spirit of the invention.

1. An apparatus for tracking the movement of an eye of a patient, comprising:
a first light source;
optical components for directing light generated by said first light source to said eye along a first light source path;
a tracking light source;
wherein tracking light generated by said tracking light source propagates to said eye along a tracking light source path;
wherein portions of said tracking light source path and said first light source path that are adjacent said eye are nearly angularly co-incident with one another;
optics for imaging on a detector plane tracking light reflected from said eye to thereby form a reflected tracking light image of a cornea and a sclera of said eye; and
a detector array at said detector plane.

2. The apparatus of claim 1 wherein portions of said tracking light source path and said first light source path that are adjacent said eye form an angle on the order of 8 degrees relative to one another.

3. The apparatus of claim 1 further comprising a video camera detector, and said video camera detector and said detector array detector do not receive light along the same optical path.

4. The apparatus of claim 1 further comprising means for generating a null signal when said image is centered on said detector array.

5. The apparatus of claim 1 further comprising means for generating a null signal when first light source path is aligned with said eye.

6. The apparatus of claim 1 further comprising means for generating an error signal proportional to the deviation of said image from a center of said detector array.

7. The apparatus of claim 1 further comprising means for maintaining an approximately centered condition between an optical axis of said laser beam and said eye based on an error signal generated by said detector array.

8. The apparatus of claim 1 further comprising a mirror for reflecting and light generated by said first light source.

9. The apparatus of claim 8 further comprising a means for deflecting said mirror.

10. The apparatus of claim 1, wherein said tracking light has a wavelength of approximately 0.8 um to approximately 1.0 .um and said light generated by said first light source has a mid-infrared wavelength.

11. The apparatus of claim 1 further comprising means for modulating intensity of said tracking light at a predefined frequency.

12. The apparatus of claim 1 further comprising one or more red filters in front of said detector array.

13. The apparatus of claim 1 further comprising one or more infra red filters in front of said detector array.

14. The apparatus of claim 1 wherein said tracking light source generates is a laser capable of generating pulses having an inter pulse duration of less than ten milliseconds.

15. The apparatus of claim 1 further comprising means for synchronizing said detector array to a frequency.

16. The apparatus of claim 1 wherein said first light source is a laser capable of ablating or photo spallating tissue of said eye.

17. The apparatus of claim 1 wherein said detector array comprises a plurality of detector elements; and
wherein each one of said plurality of detector elements is located in said detector plane at a position that does not image a center region of said cornea when said eye is aligned with said first path.

18. An method for tracking the movement of an eye of a patient, comprising:
directing light generated by a first light source to said eye along a first light source path;
propagating tracking light to said eye along a tracking light source path;
wherein portions of said tracking light source path and said first light source path that are adjacent said eye are nearly angularly co-incident with one another;
forming a reflected tracking light image of a cornea and a sclera of said eye at a detector plane; and
detecting reflected tracking light in said detector plane with a detector array.

19. The method of claim 18 wherein portions of said tracking light source path and said first light source path that are adjacent said eye form an angle on the order of 8 degrees relative to one another.

20. The method of claim 18 further comprising receiving light in a video camera detector, and said video camera detector and said detector array do not receive light along the same optical path.

21. The method of claim 18 further comprising means for generating a null signal when said image is centered on said detector array.

22. The method of claim 18 further comprising means for generating a null signal when first light source path is aligned with said eye.

23. The method of claim 18 further comprising means for generating an error signal proportional to the deviation of said image from a center of said detector array.

24. The method of claim 18 further comprising means for maintaining an approximately centered condition between an optical axis of said laser beam and said eye based on an error signal generated by said detector array.

25. The method of claim 18 further comprising a mirror for reflecting and light generated by said first light source.

26. The apparatus of claim 25 further comprising a means for deflecting said mirror.

27. The method of claim 18, wherein said tracking light has a wavelength of approximately 0.8 um to approximately 1.0 .um and said light generated by said first light source has a mid-infrared wavelength.

28. The method of claim 18 further comprising means for modulating intensity of said tracking light at a predefined frequency.

29. The method of claim 18 further comprising one or more red filters in front of said detector array.

30. The method of claim 18 further comprising one or more infra red filters in front of said detector array.

31. The method of claim 18 wherein said tracking light source generates is a laser capable of generating pulses having an inter pulse duration of less than ten milliseconds.

32. The method of claim 18 further comprising means for synchronizing said detector array to a frequency.

33. The method of claim 18 wherein said first light source is a laser capable of ablating or photo spallating tissue of said eye.

34. The method of claim 18 wherein said detector array comprises a plurality of detector elements each of which is located in said detector plane at a position that does not image a center region of said cornea when said eye is aligned with said first path.

35. A mid-infrared laser system for performing a laser surgical procedure on a tissue, said system comprising:
a laser source for producing a pump beam;
a nonlinear crystal for parametrically converting the pump beam into an idler beam and a signal beam, said idler beam having a wavelength in the mid-infrared range corresponding approximately to an absorption peak of said tissue; and
a mirror for directing said idler beam onto said tissue to remove portions of said tissue to ablate said tissue; and
wherein said system is designed to propagate said idler beam from said mirror to said tissue without said idler beam contacting any other optical elements.

36. The system of claim 35, wherein said pump beam has a pulse duration of less than 50 ns.

37. The system pf claim 35, wherein said idler beam has energy output of at least 5 mJ.

38. The system of claim 35 further comprising a scanner-deflector including said mirror for scanning said idler beam.

39. The system of claim 35, wherein said system is designed for producing a refractive correction.

40. The system of claim 35, wherein said pump beam has a pulse duration of less than about 25 nanoseconds.

41. The system of claim 35, further comprising an eye tracker to sense and compensate for movements of the eye during a procedure.

42. The system of claim 35, wherein said laser source is coupled to said mirror by a decoupled laser delivery system.

43. The system of claim 35, wherein said laser source is an erbium YAG laser.

44. The system of claim 35, wherein said laser source is a solid state laser emitting radiation in the range of approximately 1 to 2 microns.

45. The system of claim 35, wherein said laser source is designed to produce pulses, and energy in one of said pulses is between about 5 mJ and about 30 mJ.

46. The system of claim 35, wherein said system is designed to produce a spot size on an anterior surface of said tissue having a dimension in the range of about 0.3 mm to about 2 mm.

47. The system of claim 35, further comprising a corneal topography device for evaluating the shape of an anterior surface of said tissue.

48. The system of claim 35, wherein said tissue is corneal tissue, and further comprising a spatially resolved refractometer for evaluating the refraction of said corneal tissue.

49. The system of claim 35 wherein said tissue is corneal tissue.

50. A mid-infrared laser system for performing a laser surgical procedure on a tissue, said system comprising:
a laser source for producing a pump beam;
a nonlinear crystal for parametrically converting the pump beam into an idler beam and a signal beam, said idler beam having a wavelength in the mid-infrared range corresponding approximately to an absorption peak of said tissue;
structure for directing said idler beam onto said tissue to remove portions of said tissue; and
wherein said system comprises optical elements, and said structure is designed to form said idler beam into an image of an aperture of one of said optical elements at an anterior surface of said tissue.

51. The system of claim 50, wherein said pump beam has a pulse duration of less than 50 ns.

52. The system pf claim 50, wherein said idler beam has energy output of at least 5 mJ.

53. The system of claim 50, wherein said structure comprises a scanner-deflector.

54. The system of claim 50, wherein said aperture is an output aperture of a lens.

55. The system of claim 50, wherein said aperture is an output aperture of one or more optical fibers.

56. The system of claim 50, wherein said system is designed for producing a refractive correction.

57. The system of claim 50, wherein said pump beam has a pulse duration of less than about 25 nanoseconds.

58. The system of claim 50, further comprising an eye tracker to sense and compensate for movements of the eye during a procedure.

59. The system of claim 50, wherein said laser source is coupled to said structure by a decoupled laser delivery system.

60. The system of claim 50, wherein said laser source is an erbium YAG laser.

61. The system of claim 50, wherein said laser source is a solid state laser emitting radiation in the wavelength range of approximately 1 to 2 microns.

62. The system of claim 50, wherein said laser source is designed to produce pulses, and energy in one of said pulses is between about 5 ml and about 30 mJ.

63. The system of claim 50, wherein said system is designed to produce a spot size on an anterior surface of said tissue having a dimension in the range of about 0.3 mm to about 2 mm.

64. The system of claim 50, further comprising a corneal topography device for evaluating the shape of an anterior surface of said tissue.

65. The system of claim 50, wherein said tissue is corneal tissue, and further comprising a spatially resolved refractometer for evaluating the refraction of said corneal tissue.

66. The system of claim 50 wherein said tissue is corneal tissue.

67. A medical apparatus for removing corneal tissue from an eye of a patient primarily by photo spallation, said apparatus comprising:
a laser system that produces pulses of mid-infrared radiation, wherein said infrared radiation has a wavelength approximately corresponding to a corneal absorption peak, and wherein said pulses have a duration of at least about 1 nanosecond;
a scanner-deflection means to direct the pulsed radiation across an area of said corneal tissue in a predefined pattern to remove portions of said corneal tissue;
wherein said scanner-deflection means includes a mirror designed to redirect pulses towards said corneal tissue; and
said laser system includes no elements between said mirror and said corneal tissue.

68. A medical apparatus for removing corneal tissue from an eye of a patient primarily by photo spallation, said apparatus comprising:
a laser system that produces pulses of mid-infrared radiation, wherein said infrared radiation has a wavelength approximately corresponding to a corneal absorption peak, and wherein said pulses have a duration of at least about 1 nanosecond; and
a scanner-deflection means to direct the pulsed radiation across an area of said corneal tissue in a predefined pattern to remove portions of said corneal tissue; and
wherein said laser system is designed to image an output aperture of an optical element of said apparatus on said corneal tissue.

69. A method for using a mid-infrared laser system for performing a laser surgical procedure on a tissue, comprising:
producing a pump beam using a laser source;
parametrically converting the pump beam into an idler beam and a signal beam, using a nonlinear crystal, said idler beam having a wavelength in the mid-infrared range corresponding approximately to an absorption peak of said tissue; and
directing said idler beam onto said tissue using a mirror, to remove portions of said tissue to ablate said tissue; and
propagating said idler beam from said mirror to said tissue without said idler beam contacting any other optical elements.

70. The method of claim 69, wherein said pump beam has a pulse duration of less than 50 ns.

71. The method of claim 69, wherein said idler beam has energy output of at least 5 mJ.

72. The method of claim 69 further comprising scanning said idler beam using a scanner-deflector including said mirror.

73. The method of claim 69, further comprising producing a refractive correction.

74. The method of claim 69, wherein said pump beam has a pulse duration of less than about 50 nanoseconds.

75. The system of claim 69, further comprising sensing and compensating for movements of the eye during a procedure, using an eye tracker.

76. The method of claim 69, decoupling said laser source and said mirror using a decoupled laser delivery system.

77. The method of claim 69, wherein said laser source is an erbium YAG laser.

78. The method of claim 69, wherein said laser source is a solid state laser emitting radiation in the wavelength range of approximately 1 to 2 microns.

79. The method of claim 69, wherein said laser source is designed to produce pulses, and energy of one of said pulses is between about 5 mJ and about 30 mJ.

80. The method of claim 69, further comprising producing a spot size on an anterior surface of said tissue having a dimension in the range of about 0.3 mm to about 2 mm.

81. The method of claim 69, evaluating the shape of an anterior surface of said tissue using a corneal topography device.

82. The method of claim 69, wherein said tissue is corneal tissue, and further comprising evaluating the refraction of said corneal tissue using a spatially resolved refractometer.

83. The method of claim 69 wherein said tissue is corneal tissue.

84. A method for performing a laser surgical procedure on a tissue using a mid-infrared laser system, comprising:
producing a pump beam using a laser source;
parametrically converting the pump beam into an idler beam and a signal beam using a nonlinear crystal, said idler beam having a wavelength in the mid-infrared range corresponding approximately to an absorption peak of said tissue;
directing said idler beam onto said tissue using structure, to remove portions of said tissue; and
wherein said system comprises optical elements, and said structure is designed to form said idler beam into an image of an aperture of one of said optical elements at an anterior surface of said tissue.

85. The method of claim 84, wherein said pump beam has a pulse duration of less than 50 ns.

86. The method pf claim 84, wherein said idler beam has energy output of at least 5 mJ.

87. The method of claim 84, wherein said structure comprises a scanner-deflector.

88. The method of claim 84, wherein said aperture is an output aperture of a lens.

89. The method of claim 84, wherein said aperture is an output aperture of one or more optical fibers.

90. The method of claim 84, further comprising producing a refractive correction.

91. The method of claim 84, wherein said pump beam has a pulse duration of less than about 50 nanoseconds.

92. The method of claim 84, further comprising sensing and compensating for movements of said tissue during a procedure, using an eye tracker.

93. The method of claim 84, decoupling said laser source and said structure using a decoupled laser delivery system.

94. The method of claim 84, wherein said laser source is an erbium YAG laser.

95. The method of claim 84, wherein said laser source is a solid state laser emitting radiation in the wavelength range of approximately 1 to 2 microns.

96. The method of claim 84, wherein said pump beam is designed to produce pulses, and energy of one of said pulses is between about 5 mJ and about 30 mJ.

97. The method of claim 84, wherein said system is designed to produce a spot size on an anterior surface of said tissue having a dimension in the range of about 0.3 mm to about 2 mm.

98. The method of claim 84, further comprising using a corneal topography device.

99. The method of claim 84, wherein said tissue is corneal tissue, and further comprising evaluating the refraction of said corneal tissue using a spatially resolved refractometer.

100. The method of claim 84 wherein said tissue is corneal tissue.
